(19) United States
(12) Patent Application Publication
(10) Pub. No.: US 2023/0131989 A1
(43) Pub. Date: Apr. 27, 2023

(54) CANNABINOID COMPOSITIONS AND METHODS OF USING FOR THE TREATMENT OF NON-EOSINOPHILIC INFLAMMATION AND INFLAMMATORY DISORDERS

(71) Applicant: MEDTERRA PHARMA LLC, Irvine, CA (US)

(72) Inventor: Matthew Halpert, Irvine, CA (US)

(21) Appl. No.: 17/874,910

(22) Filed: Jul. 27, 2022

Related U.S. Application Data
(63) Continuation-in-part of application No. 17/389,302, filed on Jul. 29, 2021.
(60) Provisional application No. 63/058,302, filed on Jul. 29, 2020.

(51) Int. Cl. A61K 9/127; A61K 31/05; A61P 11/06; A61P 29/00

(52) U.S. Cl. CPC A61K 9/127; A61K 31/05; A61P 11/06; A61P 29/00

(57) ABSTRACT

Disclosed herein are compositions for treating a non-eosinophilic inflammation or inflammatory disorder. The compositions comprise a cannabinoid encapsulated in a drug vehicle. The compositions can be aerosolized for delivery into all pulmonary spaces of a subject in need thereof. The compositions can be used to treat pulmonary inflammatory conditions, including severe asthma (SA), non-eosinophilic asthma, and neutrophilic asthma.

### CROSS-REFERENCE TO RELATED APPLICATIONS

This application is a continuation-in-part of U.S. application Ser. No. 17/389,302, filed Jul. 29, 2021, which claims priority to U.S. Provisional Application No. 63/058,302, filed Jul. 29, 2020, the contents of all of which are incorporated herein by reference in their entirety.

### FIELD OF THE INVENTION

The present invention generally relates to encapsulated cannabinoid compositions and methods of using the encapsulated cannabinoid compositions for treating non-eosinophilic inflammation and inflammatory disorders.

### BACKGROUND OF THE INVENTION

Numerous disease conditions are associated with abnormal inflammation. For instance, pulmonary inflammation and inflammatory disorders are a major problem across the US and the world. For example, about 25 million Americans suffer from Asthma, with about 10% of those being considered in “dire” conditions. About 4,000 US patients die each year from an Asthmatic attack, a relatively high number for something many consider to be completely benign. Looking globally, there are over 300 million people suffering from this condition.

The current treatment modalities for pulmonary inflammation and inflammatory disorders are sub-par to say the least. Global reduction of “inflammation” by constant use of steroids and non-steroidal anti-inflammatories is an insufficient approach as limited amounts of drug will make it through the oral or topical routes to the lung spaces, will simultaneously tax the liver and other organs leading to toxicity-related outcomes, and have been shown to not be effective at reducing this pulmonary inflammation even when directly applied. Constant use of biologics such as anti-TNFα therapy to reduce inflammation is an insufficient approach as limited amounts of drug will make it through the oral or topical routes to the lung spaces. Further, biologics often have to be injected and are expensive (barriers to common and chronic use), will allow for more opportunistic infections, and have yet to be demonstrated to be effective. About 2 million Americans a year fail to respond to these treatments and end up hospitalized with no real treatment option (a.k.a. orphan disorder).

Accordingly, there is an urgent need in the art for effective treatment options of pulmonary inflammation and inflammation disorders such as steroid-unresponsive uncontrolled asthma without the disadvantages associated with currently available treatment methods.

### SUMMARY OF THE INVENTION

One aspect of the instant disclosure encompasses a method of treating a non-eosinophilic inflammation or inflammatory disorder in a subject in need thereof. The method comprises administering a therapeutically effective amount of a composition comprising cannabidiol (CBD) to the subject, wherein the composition is encapsulated in a liposomal drug delivery system.

The inflammation or inflammatory disorder can be a pulmonary inflammation or inflammatory disorder. For instance, the pulmonary inflammation or inflammatory disorder is asthma, chronic obstructive pulmonary disease (COPD), allergic bronchopulmonary aspergillosis, hypersensitivity pneumonia, eosinophilic pneumonia, emphysema, bronchitis, allergic bronchitis bronchiectasis, cystic fibrosis, tuberculosis, hypersensitivity pneumonitis, occupational asthma, sarcoid, reactive airway disease syndrome, interstitial lung disease hypereosinophilic syndrome, rhinitis, sinusitis, exercise-induced asthma, pollution-induced asthma, cough variant asthma, parasitic lung disease, bacterial infections, respiratory syncytial virus (RSV) infection, parainfluenza virus (PIV) infection, rhinovirus (RV) infection or adenovirus infection, or any combination thereof. In some aspects, the pulmonary inflammation or inflammatory disorder is non-eosinophilic asthma. In other aspects, the pulmonary inflammation or inflammatory disorder is a neutrophilic pulmonary inflammation or inflammatory disorders. In yet other aspects, the pulmonary inflammation or inflammatory disorder is neutrophilic asthma. In additional aspects, the pulmonary inflammation or inflammatory disorder is severe asthma (SA). In some aspects, the pulmonary inflammation or inflammatory disorder is steroid-resistant asthma.

The method can further comprise aerosolizing the composition before administering to pulmonary spaces of the subject. When the composition is aerosolized, the therapeutically effective amount of CBD is in a unit dose form, and the method comprises administering the CBD by aerosolizing the unit dose of the composition to the pulmonary spaces. The unit dose can be present in and/or delivered from a device suitable for aerosolizing the unit dose into the pulmonary spaces of the subject. In some aspects, the unit dose comprises from about 0.6 mg to about 0.9 mg of CBD to a human subject. In some aspects, the pulmonary inflammation or inflammatory disorder is SA or steroid-resistant asthma, wherein the method comprises administering a unit dose form of the composition to pulmonary spaces of the subject, and wherein the unit dose comprises from about 0.6 mg to about 0.9 mg of CBD.

The inflammatory disorder can also be osteoarthritis. When the inflammatory disorder is osteoarthritis, the method can comprise orally administering from about 90 mg to about 110 mg of CBD to a human subject. In some aspects, about 20 mg/kg of CBD are administered to the human subject per day.

Another aspect of the instant disclosure encompasses a method of treating osteoarthritis in a subject in need thereof. The method comprises orally administering a composition comprising a therapeutically effective amount of cannabidiol (CBD) encapsulated in a liposomal drug delivery system. The therapeutically effective amount comprises from about 0.6 mg to about 0.9 mg of CBD.

Yet another aspect of the instant disclosure encompasses a method of preventing exacerbation of eosinophilic inflammation or inflammatory disorder in a subject in need thereof. The method comprises preventing administration of a pharmaceutical composition comprising cannabidiol (CBD) to the subject. In some aspects, the CBD is encapsulated in a liposomal drug delivery system.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 is an illustration depicting penetration of various sized liposomally encapsulated cannabidiol administered with a nebulizer into airways as quantified in the graph showing % distribution of liposomes. Mass Mean Aerodynamic Diameter Range: 0.4-2μ(>70%). Data presented are from drug extracted and quantified by HPLC-MS/MS. The liposomes were quantified by HPLC-MS/MS. Andersen Cascade Impactor (ACI) plates

FIG. 2 are electron microscopy images of CBD-D liposomes.

FIG. 3A schematically depicts the study design for a mycosis-induced murine model of eosinophilic asthma.

FIG. 3B graphically displays the changes in airway resistance (proxy measure of lung damage) to increasing doses of Aspergillus niger conidia in challenged, non-challenged and treated or untreated mice.

FIG. 4A schematically depicts the study design for a mycosis- and LPS-induced murine model of neutrophilic asthma.

FIG. 4B graphically displays the changes in airway resistance (proxy measure of lung damage) to increasing doses of A. niger conidia and LPS in challenged, non-challenged and treated or untreated mice.

FIG. 4C graphically displays the changes in airway resistance (proxy measure of lung damage) to increasing doses of A. niger conidia and LPS in challenged, non-challenged and treated or untreated mice.

FIG. 4D graphically displays the changes in airway resistance (proxy measure of lung damage) to increasing doses of A. niger conidia and LPS in challenged, non-challenged and treated or untreated mice.

FIG. 5 is a plot showing the effects of CBD-D on neutrophil levels in the mice described in this example.

FIG. 6 is a plot showing neutrophil levels in mice depleted or undepleted of neutrophils.

FIG. 7 is a plot depicting the effect of CBD delivered by CBD-D in challenged mice in the murine neutrophilic model of asthma on cytokine levels (upper panel). The lower panel depicts the function of each cytokine during development of inflammation.

### DETAILED DESCRIPTION

The present disclosure is based in part on the surprising discovery that cannabinoids can selectively treat non-eosinophilic inflammation and inflammatory disorders but not eosinophilic disorders. In fact, the inventors discovered that cannabinoids can even exacerbate an eosinophilic inflammation and inflammatory disorder. The inventors discovered that cannabinoids can effectively treat acute inflammation, arthritis, and pulmonary inflammation.

The inventors also discovered that cannabinoids, when encapsulated, can significantly improve efficacy of administered cannabinoids against inflammation and inflammatory disorders. Significantly lower doses of the cannabinoids can be used when encapsulated to effectively treat inflammation, when compared to naked cannabinoids. Importantly, encapsulated can deliver therapeutically effective amounts of the cannabinoid into pulmonary spaces where the cannabinoid was surprisingly found to be effective in treating pulmonary inflammation and pulmonary inflammation disorders. The compositions and methods of the instant disclosure can deliver therapeutically effective amounts of the cannabinoids directly into all pulmonary spaces, thereby avoiding the toxicity and other disadvantages associated with currently available inhalation and systemic delivery modalities of cannabinoids.

### I. Composition

One aspect of the present disclosure encompasses pharmaceutical compositions comprising an encapsulated cannabinoid. The compositions can be administered into all pulmonary spaces. The cannabinoids are encapsulated in a drug delivery vehicle, and the can be administered topically, parenterally, or can be aerosolized for delivery by inhalation. Cannabinoids, encapsulated cannabinoids, and compositions of encapsulated cannabinoids are described further below.

(a) Cannabinoids

A composition of the instant disclosure comprises a cannabinoid. Cannabis and its constituents are increasingly being recognized as bona fide pharmacologic agents with significant therapeutic potential. The pharmacological activity can be related to the role in which the ubiquitous endocannabinoid system plays in many physiological and pathophysiological processes. For example, cannabidiol (CBD), the major non-tetrahydrocannabinol (THC) constituent of cannabis, can exert numerous biological effects through several different receptors and signaling pathways, including anti-inflammatory effects in both acute and chronic conditions. However, use of cannabinoids for treating disease conditions faces major obstacles. For instance, one major obstacle for use of CBD include it's high degree of insolubility and low bioavailability. Further, the rate of metabolism of CBD by the p450 enzymes in the liver limits the amount of effective CBD in the body.

When used for treatment of pulmonary inflammation and inflammatory disorders, tracking of CBD from the gastrointestinal tract or circulatory system to the pulmonary spaces is also inefficient, regardless of metabolic or absorption rates. Combined, these factors render administration of CBD through the oral, parenteral, and topical routes inefficient. Administration through inhalation, a seemingly more direct route of administration for pulmonary disease conditions, is also inefficient especially to the lower airway and especially during inflammation that further compromise the delivery of any anti-inflammatory to the lower airways. Further, inhalation and smoking of cannabis or CBD is harmful to the lung spaces, can cause destruction and inflammation, and potentially even death. Surprisingly, the inventors discovered through extensive experimentation that encapsulating cannabinoids can overcome all these obstacles and can efficiently deliver the cannabinoids to target tissues including throughout the lungs. Encapsulation of cannabinoids can be as described in Section I(b) herein below.

Any cannabinoid capable of providing anti-inflammatory activity against inflammation or inflammatory disorders that are not associated with inflammation caused by or exacerbated by eosinophils. As used herein, the term “cannabinoid” refers to a chemical substance that interacts with cannabinoid receptors of the body and brain, regardless of structure or origin, or analogs of naturally occurring cannabinoids, including naturally occurring phytocannabinoids. Phytocannabinoids are cannabinoids produced by a species of the Cannabis plant. More than 80 cannabinoids and more than 300 non-cannabinoid phytoconstituents such as terpenes and terpenoids have been identified in Cannabis plants to date. These cannabinoids are abundant in the viscous resin that is produced by glandular structures in the Cannabis plant called trichomes. This resin is also rich in terpenes, which are responsible for the characteristic smell of the Cannabis plant. A cannabimimetic agent is a composition characterized as having at least 50% of the psychoactive effect of Δ⁸-tetrahydrocannabinol. The mimetic may differ from Δ⁸-tetrahydrocannabinol in structure, pattern of side group substitution, or both.

Phytocannabinoids are known to occur in several plant species besides Cannabis. These include Echinacea purpurea, Echinacea angustifolia, Acmella oleracea, Helichrysum umbraculigerum, and Radula marginata. The best-known cannabinoids that are not derived from Cannabis are the lipophilic alkamides (alkylamides) from Echinacea species, most notably the cis/trans isomers dodeca-2E,4E,8Z,10E/Z-tetraenoic-acid-isobutylamide. At least 25 different alkylamides have been identified, and some of them have shown affinities to the CB2-receptor. In some Echinacea species, cannabinoids are found throughout the plant structure, but are most concentrated in the roots and flowers. The Kava plant has significant affinity to the CB1 receptor. Tea (Camellia sinensis) catechins have an affinity for human cannabinoid receptors. A widespread dietary terpene, beta-caryophyllene, a component from the essential oil of Cannabis and other medicinal plants, has also been identified as a selective agonist of peripheral CB2-receptors, in vivo. Black truffles contain anandamide. Perrottetinene, a moderately psychoactive cannabinoid, has been isolated from different Radula varieties.

A large number of cannabinoids have been grouped into classes based on similarities in their general (or Markush) chemical structure. Typical groups of compounds can include, without limitation, naphthoylindoles, phenylacetylindoles, benzoylindoles, cyclohexylphenols, naphthylmethylindoles, naphthoylpyrroles, naphthylmethylindenes, indole-3-carboxamides, indole-3-carboxylates, indazole-3-carboxamides and sometimes others, each with specific substitutions on specific atoms of the molecule.

Non limiting examples of cannabinoids include tetrahydrocannabinol (THC), cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), tetrahydrocannabivarinic acid (THCVA), any combination thereof, any natural or synthetic modification or derivative thereof, salts and acids thereof, or any natural or synthetic molecule that interacts directly or indirectly with cannabinoid receptors of the body and brain and that have similar effects to those produced by a Cannabis plant.

Phytocannabinoids of the instant disclosure can be obtained by extracting from a plant. The cannabinoids can be highly purified or can comprise other plant constituents such as flavonoid compounds, terpenes, terpenoid, and synthetic, semisynthetic or highly purified versions of any such constituent. Phytocannabinoids can be obtained as either the neutral (decarboxylated form) or the carboxylic acid form depending on the method used to extract the cannabinoids. For example, it is known that heating the carboxylic acid form will cause most of the carboxylic acid form to decarboxylate into the neutral form. “Highly purified cannabinoids” are defined as cannabinoids that have been extracted from the cannabis plant and purified to the extent that other cannabinoids and non-cannabinoid components that are co-extracted with the cannabinoids have been removed, such that the highly purified cannabinoid is greater than or equal to about 95% (w/w) pure.

Cannabinoids of the instant disclosure can also be chemically synthesized in the laboratory. See, Stedman, Medical Dictionary, pg. III, Williams & Wilkins, Baltimore, Md. (1987). Cannabinoids synthesized by the hemp plant include, but are not limited to, cannabinol, cannabidiol, cannabinolic acid, cannabigerol, cannabicyclol, and several isomers of tetrachydrocannabinol (THC). See, Goodman and Gilman, The Pharmacological Basis of Therapeutics, 6th Ed., pp. 560-563, MacMillan Publishing, New York, N.Y. (1983).

Cannabinoids of the instant disclosure can be analogs of naturally occurring cannabinoids. Historically, cannabinoid analogs are based on the structure of phytocannabinoids, and a large number of analogs have been produced and tested. Newer compounds are no longer related to natural cannabinoids or are based on the structure of the endogenous cannabinoids. The term “neocannabinoid” can distinguish these designer drugs from chemically synthesized phytocannabinoids (THC or CBD obtained by chemical synthesis) or endocannabinoids.

Non-limiting examples of synthetic cannabinoids are listed in Table 1 below.

TABLE 1
CANNABIGEROL-TYPE (CBG)

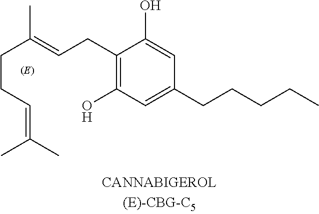 [SMILES: CCCCCc1cc(O)c(C/C=C(\C)CCC=C(C)C)c(O)c1]

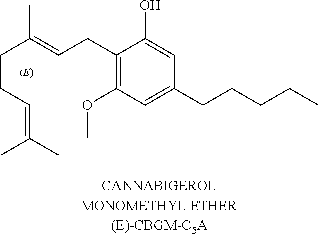 [SMILES: CCCCCc1cc(O)c(C/C=C(\C)CCC=C(C)C)c(OC)c1]

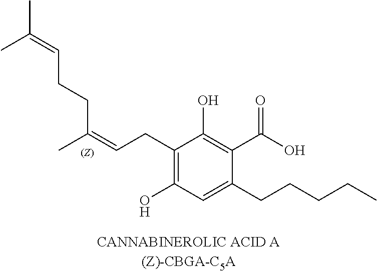 [SMILES: CCCCCc1cc(O)c(C/C=C(/C)CCC=C(C)C)c(O)c1C(=O)O]

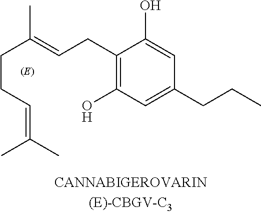 [SMILES: CCCc1cc(O)c(C/C=C(\C)CCC=C(C)C)c(O)c1]

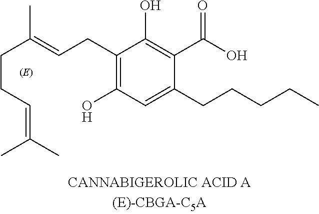 [SMILES: CCCCCc1cc(O)c(C/C=C(\C)CCC=C(C)C)c(O)c1C(=O)O]

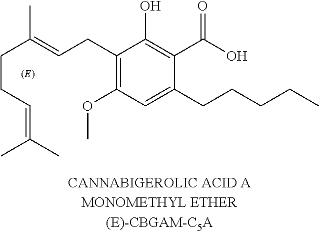 [SMILES: CCCCCc1cc(OC)c(C/C=C(\C)CCC=C(C)C)c(O)c1C(=O)O]

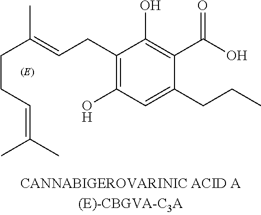 [SMILES: CCCc1cc(O)c(C/C=C(\C)CCC=C(C)C)c(O)c1C(=O)O]

CANNABICHROMENE-TYPE (CBC)

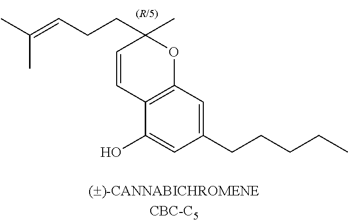 [SMILES: CCCCCc1cc(O)c2c(c1)OC(C)(CCC=C(C)C)C=C2]

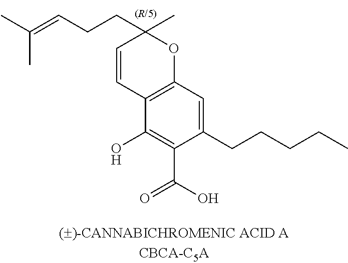 [SMILES: CCCCCc1cc2c(c(O)c1C(=O)O)C=CC(C)(CCC=C(C)C)O2]

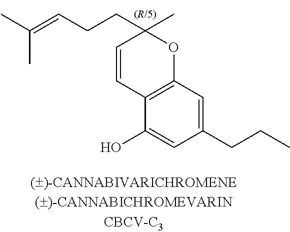 [SMILES: CCCc1cc(O)c2c(c1)OC(C)(CCC=C(C)C)C=C2]

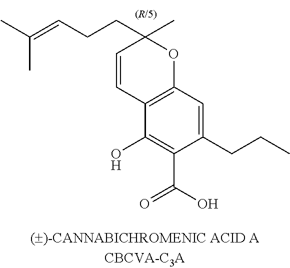 [SMILES: CCCc1cc2c(c(O)c1C(=O)O)C=CC(C)(CCC=C(C)C)O2]

CANNABIDIOL-TYPE (CBD)

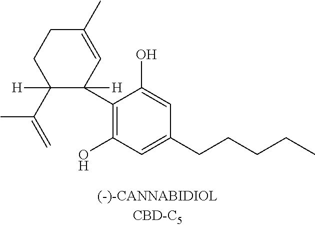 [SMILES: C=C(C)C1CCC(C)=CC1c1c(O)cc(CCCCC)cc1O]

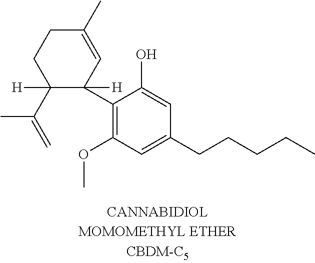 [SMILES: C=C(C)C1CCC(C)=CC1c1c(O)cc(CCCCC)cc1OC]

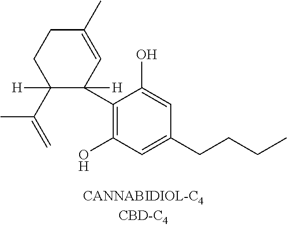 [SMILES: C=C(C)C1CCC(C)=CC1c1c(O)cc(CCCC)cc1O]

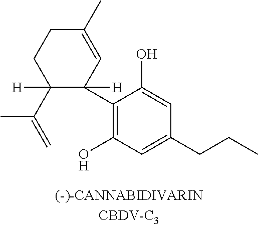 [SMILES: C=C(C)C1CCC(C)=CC1c1c(O)cc(CCC)cc1O]

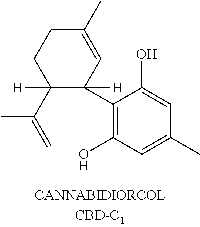 [SMILES: C=C(C)C1CCC(C)=CC1c1c(O)cc(C)cc1O]

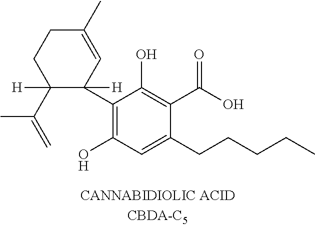 [SMILES: C=C(C)C1CCC(C)=CC1c1c(O)cc(CCCCC)c(C(=O)O)c1O]

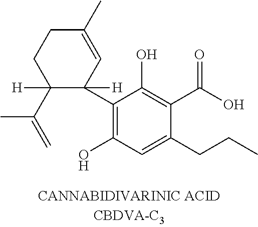 [SMILES: C=C(C)C1CCC(C)=CC1c1c(O)cc(CCC)c(C(=O)O)c1O]

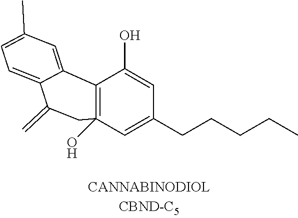

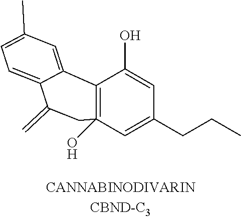

TETRAHYDROCANNABINOL-TYPE (THC)

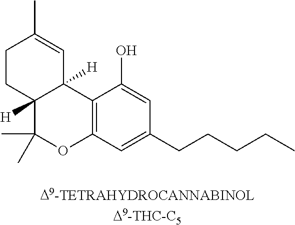 [SMILES: CCCCCc1cc(O)c2c(c1)OC(C)(C)[C@@H]1CCC(C)=C[C@@H]21]

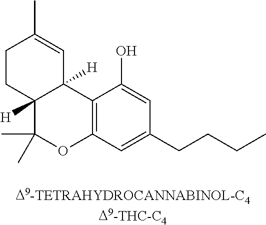 [SMILES: CCCCc1cc(O)c2c(c1)OC(C)(C)[C@@H]1CCC(C)=C[C@@H]21]

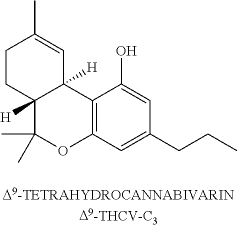 [SMILES: CCCc1cc(O)c2c(c1)OC(C)(C)[C@@H]1CCC(C)=C[C@@H]21]

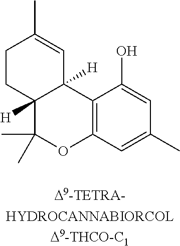

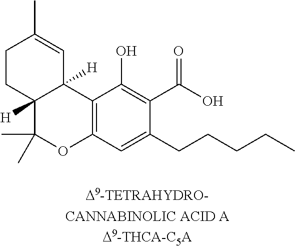 [SMILES: CCCCCc1cc2c(c(O)c1C(=O)O)[C@@H]1C=C(C)CC[C@H]1C(C)(C)O2]

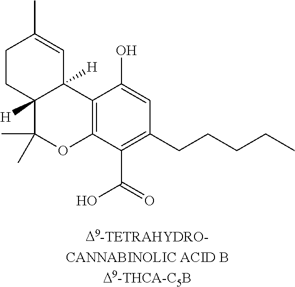 [SMILES: CCCCCc1cc(O)c2c(c1C(=O)O)OC(C)(C)[C@@H]1CCC(C)=C[C@@H]21]

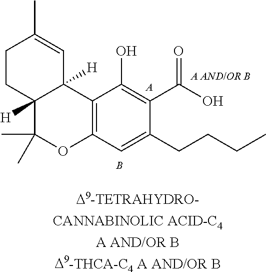

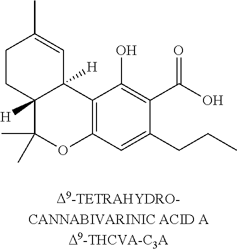 [SMILES: CCCc1cc2c(c(O)c1C(=O)O)[C@@H]1C=C(C)CC[C@H]1C(C)(C)O2]

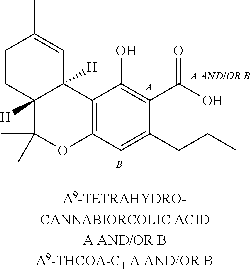

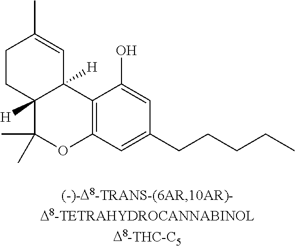 [SMILES: CCCCCc1cc(O)c2c(c1)OC(C)(C)[C@@H]1CCC(C)=C[C@@H]21]

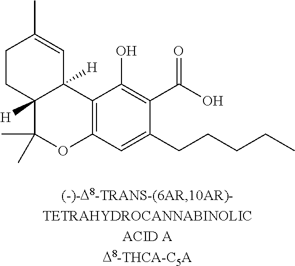 [SMILES: CCCCCc1cc2c(c(O)c1C(=O)O)[C@@H]1C=C(C)CC[C@H]1C(C)(C)O2]

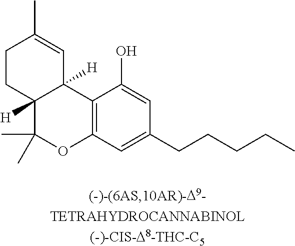 [SMILES: CCCCCc1cc(O)c2c(c1)OC(C)(C)[C@@H]1CCC(C)=C[C@@H]21]

CANNABINOL-TYPE (CBN)

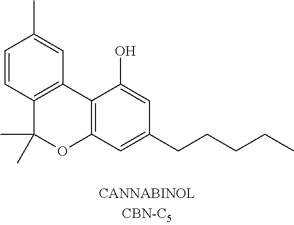 [SMILES: CCCCCc1cc(O)c2c(c1)OC(C)(C)c1ccc(C)cc1-2]

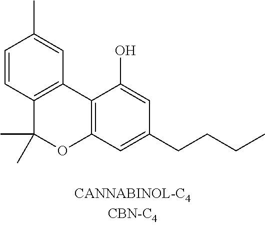 [SMILES: CCCCc1cc(O)c2c(c1)OC(C)(C)c1ccc(C)cc1-2]

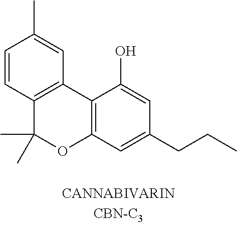 [SMILES: CCCc1cc(O)c2c(c1)OC(C)(C)c1ccc(C)cc1-2]

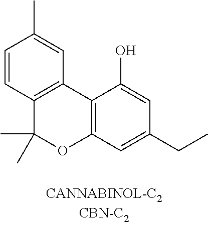 [SMILES: CCc1cc(O)c2c(c1)OC(C)(C)c1ccc(C)cc1-2]

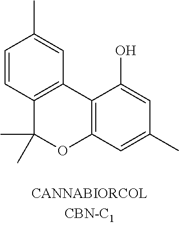

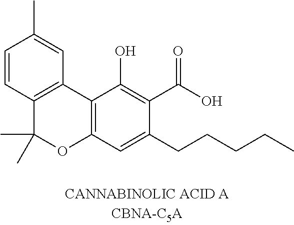 [SMILES: CCCCCc1cc2c(c(O)c1C(=O)O)-c1cc(C)ccc1C(C)(C)O2]

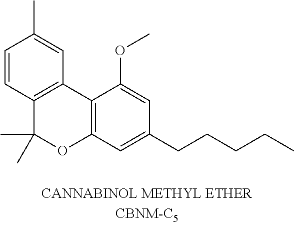 [SMILES: CCCCCc1cc(OC)c2c(c1)OC(C)(C)c1ccc(C)cc1-2]

CANNABITRIOL-TYPE (CBT)

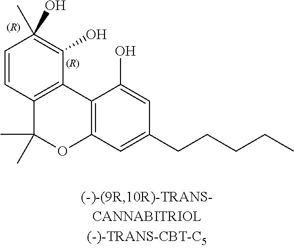

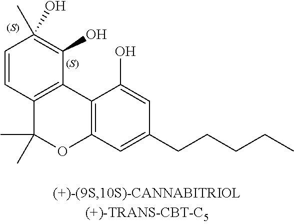

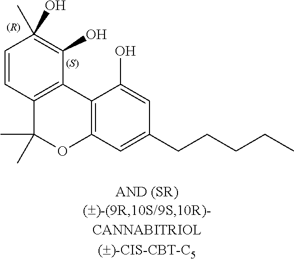

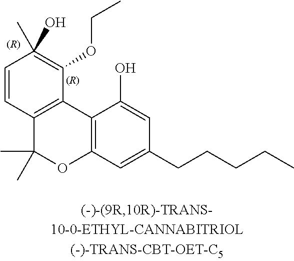

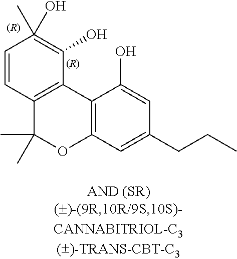

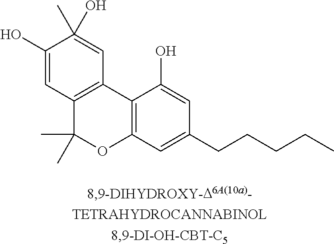

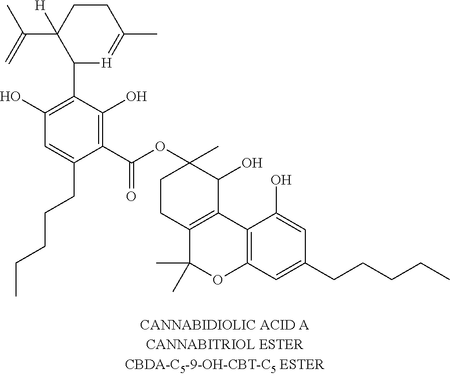

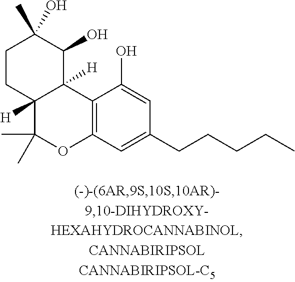 [SMILES: CCCCCc1cc(O)c2c(c1)OC(C)(C)[C@@H]1CC[C@](C)(O)[C@@H](O)[C@@H]21]

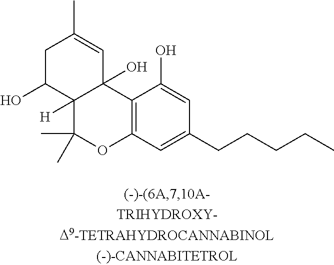 [SMILES: CCCCCc1cc(O)c2c(c1)OC(C)(C)C1C(O)CC(C)=CC21O]

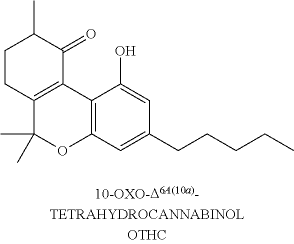 [SMILES: CCCCCc1cc(O)c2c(c1)OC(C)(C)C1=C2C(=O)C(C)CC1]

CANNABIELSOIN-TYPE (CBE)

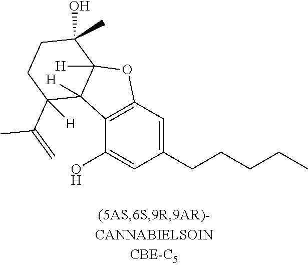

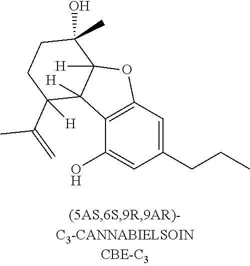

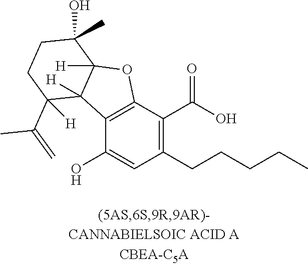

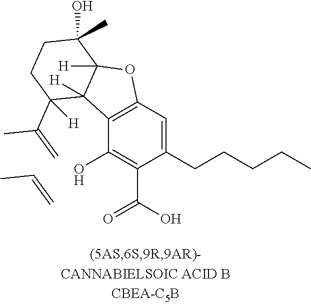

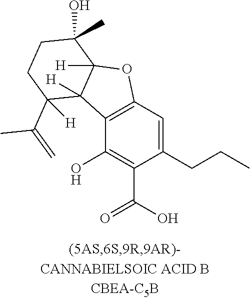

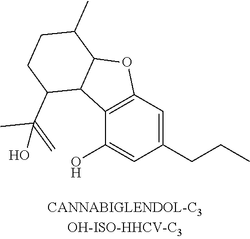

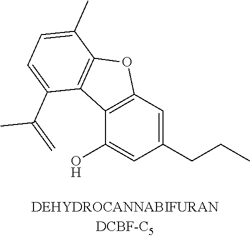 [SMILES: C=C(C)c1ccc(C)c2oc3cc(CCC)cc(O)c3c12]

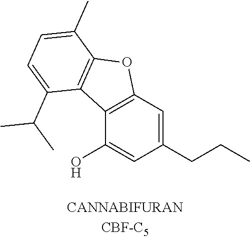 [SMILES: CCCc1cc(O)c2c(c1)oc1c(C)ccc(C(C)C)c12]

ISOCANNABINOIDS

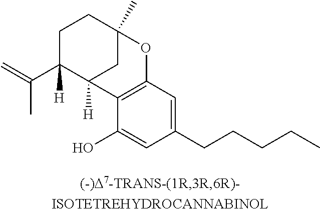 [SMILES: C=C(C)[C@@H]1CC[C@]2(C)C[C@H]1c1c(O)cc(CCCCC)cc1O2]

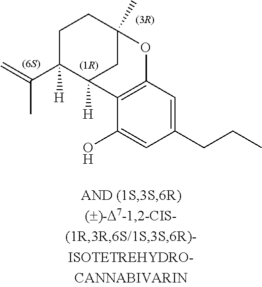 [SMILES: C=C(C)[C@H]1CC[C@]2(C)C[C@H]1c1c(O)cc(CCC)cc1O2]

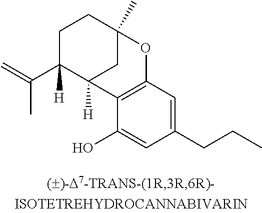 [SMILES: C=C(C)[C@@H]1CC[C@]2(C)C[C@H]1c1c(O)cc(CCC)cc1O2]

Medications containing natural or synthetic cannabinoids or cannabinoid analogs include: Dronabinol (Marinol), is Δ9-tetrahydrocannabinol (THC), used as an appetite stimulant, anti-emetic, and analgesic; Nabilone (Cesamet, Canemes), a synthetic cannabinoid and an analog of Marinol; Rimonabant (SR141716), a selective cannabinoid (CB1) receptor inverse agonist once used as an anti-obesity and smoking cessation drug under the proprietary name Acomplia; JWH-018, a potent synthetic cannabinoid often sold in legal smoke blends collectively known as “spice”; JWH-073; CP-55940, a synthetic cannabinoid receptor agonist many times more potent than THC; dimethylheptylpyran; HU-210, about 100 times as potent as THC; HU-211, a synthetic cannabinoid derived drug that acts on NMDA instead of endocannabinoid system; HU-331 a potential anti-cancer drug derived from cannabidiol that specifically inhibits topoisomerase II; SR144528, a CB2 receptor antagonist/inverse agonist; WIN 55,212-2, a potent cannabinoid receptor agonist; JWH-133, a potent selective CB2 receptor agonist; Levonantradol (Nantrodolum), an anti-emetic and analgesic; and AM-2201, a potent cannabinoid receptor agonist.

In some aspects, a cannabinoid of the instant disclosure is tetrahydrocannabinol (THC), cannabidiol (CBD), cannabigerol (CBG), cannabichromene (CBC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethyl ether (CBGM), cannabinerolic acid, cannabidiolic acid (CBDA), Cannabinol propyl variant (CBNV), cannabitriol (CBO), tetrahydrocannabinolic acid (THCA), tetrahydrocannabivarinic acid (THCVA), any derivatives, salts and acids thereof, or any combination thereof.

The best studied cannabinoids include tetrahydrocannabinol (THC), cannabidiol (CBD), and cannabinol (CBN). Tetrahydrocannabinol (THC) is the primary psychoactive component of the Cannabis plant. Delta-9-tetrahydrocannabinol (Δ9-THC, THC) and Delta-8-Tetrahydrocannabinol (08-THC), through intracellular CB1 activation, induce anandamide and 2-arachidonoylglycerol synthesis produced naturally in the body and brain. These cannabinoids produce the effects associated with cannabis by binding to the CB1 cannabinoid receptors in the brain. CBD is non-psychotropic. Evidence shows that CBD counteracts cognitive impairment associated with the use of cannabis. CBD has little affinity for CB1 and CB2 receptors but acts as an indirect antagonist of cannabinoid agonists. It was found to be an antagonist at the putative new cannabinoid receptor, GPR55, a GPCR expressed in the caudate nucleus and putamen. Cannabidiol has also been shown to act as a 5-HT1A receptor agonist. CBD can interfere with the uptake of adenosine, which plays an important role in biochemical processes, such as energy transfer. It may play a role in promoting sleep and suppressing arousal. CBD shares a precursor with THC and is the main phytocannabinoid in CBD-dominant Cannabis strains. CBD has been shown to play a role in preventing the short-term memory loss associated with THC. There is tentative evidence that CBD has an anti-psychotic effect, but research in this area is limited. CBD has proven to exert a beneficial effect in asthmatic models. Previous studies utilized CBD in a smokable form as many people choose to smoke (a.k.a. inhale) CBD as a quicker (3-10 min) and ‘better’ form of increasing systemic bioavailability. However, inhaled CBD can lead to a 30-65% systemic delivery, much higher than the ingestible amounts (Millar et al, 2018; Iffland, 2017; Lucas, 2018). Such high levels of systemic CBD (or other cannabinoids) is not desired when treating pulmonary inflammation and inflammatory disorders such as asthma. Instead, it is beneficial for CBD and other cannabinoids to remain in the pulmonary spaces to act on the recruited and activated immune cells. In some aspects, the cannabinoid of the instant disclosure is CBD.

(b) Encapsulation

A major obstacle to the use of cannabinoids such as CBD is high degree of insolubility and low bioavailability. Orally administered CBD has a high rate of metabolism by the p450 enzymes in the liver, thus limiting the amount of effective CBD that may make it to target sites. Encapsulation of the cannabinoids in nano-sized drug delivery systems (DDS) designed to enhance the delivery of APIs with poor pharmacokinetics and biodistribution can be used to deliver therapeutically effective amounts of the cannabinoid to various targets in the body, all while avoiding the toxicity and other disadvantages associated with topical and systemic delivery modalities of cannabinoids.

When the target is airspaces of the lungs, another major obstacle to the use of orally administered CBD is the inefficient tracking from the gastrointestinal tract or circulatory system to the pulmonary spaces, regardless of metabolic or absorption rates. For these reasons, when the route of administration is by inhalation, the oral, parenteral, and topical routes are less than ideal. As explained above, although the inhalation route would seem to provide a more direct delivery of the cannabinoids to the pulmonary spaces, the size and insolubility of cannabinoids prevent the cannabinoids from dispersing throughout the lung airways and thus from reaching the airway spaces, including to and through the lower airway. Further, the thinness of the pulmonary epithelium results in short residence of the inhaled drug in the lung and potential for systemic (adverse) effects.

The inventors discovered that encapsulating the cannabinoids in nano-sized drug delivery systems (DDS) designed to enhance the delivery of APIs with poor pharmacokinetics and biodistribution can be used to deliver therapeutically effective amounts of the cannabinoid, including into pulmonary spaces, all while avoiding the toxicity and other disadvantages associated with currently available inhalation and systemic delivery modalities of cannabinoids. Importantly, the inventors surprisingly discovered that encapsulating cannabinoids in a drug delivery system comprising a heterogeneous distribution of diameters can efficiently deliver the cannabinoid to all airways in the lungs, thereby significantly improving efficacy of the cannabinoid in treating pulmonary inflammation and inflammatory disorders. Further, the size distribution of encapsulated cannabinoids can be adjusted to target various spaces in the lungs. This is surprising because the consensus in the field of encapsulation of active pharmaceutical ingredients (APIs) is the use of monodisperse drug vehicles optimized to have a homogeneous diameter.

As used herein, the term “encapsulated” refers to an active ingredient such as cannabinoids of the instant disclosure encapsulated in a drug carrier or drug vehicle. A wide variety of drug delivery systems have been developed and studied, each of which has unique advantages and disadvantages. Non-limiting examples of drug vehicles include liposomes, polymeric micelles, microspheres, and nanoparticles among others. Different methods of attaching the drug to the carrier have been implemented, including adsorption, integration into the bulk structure, encapsulation, and covalent bonding. Different types of drug vehicles utilize different methods of attachment, and some vehicles can even implement a variety of attachment methods.

Accordingly, a cannabinoid composition of the instant disclosure comprises a cannabinoid encapsulated in a drug vehicle. In some aspects, a cannabinoid composition of the instant disclosure comprises a cannabinoid encapsulated in nanoparticles. Non-limiting examples of nanoparticles include nano diamonds, nanofibers, protein-DNA complexes, protein-drug complexes, protein-drug conjugates, erythrocytes, virosomes, and dendrimers.

In some aspects, a cannabinoid composition of the instant disclosure comprises a cannabinoid encapsulated in polymeric micelles. Polymeric micelles are drug vehicles formed by the aggregation of some amphiphilic molecules with an amphiphilic block copolymer. These vehicles form at some high concentration specific to the compounds used, called the critical micelle concentration. The addition of an amphiphilic block copolymer effectively lowers this critical micelle concentration by shifting the monomer exchange equilibrium. These vehicles lack an aqueous core makes polymeric micelles more accommodating to insoluble active ingredients such as cannabinoids.

In some aspects, a cannabinoid composition of the instant disclosure comprises a cannabinoid encapsulated in microspheres. Microspheres are hollow, micron-sized vehicles often formed via self-assembly of polymeric compounds which are most often used to encapsulate the active drug for delivery. Drug release from microspheres is often achieved by diffusion through pores in the microsphere structure or by degradation of the microsphere shell. Some assembly techniques, such as precision particle fabrication (PPF), can create microspheres capable of sustained control over drug release.

In some aspects, a cannabinoid composition of the instant disclosure comprises a cannabinoid encapsulated in liposomes. Liposomes are artificially spherical vesicles prepared from naturally-derived phospholipid. They entail one or more lipid bilayers with discrete aqueous spaces. They are well established for a range of pharmaceutical and biomedical applications with the unique capability of entrapment of both hydrophilic (polar) and hydrophobic (nonpolar) compounds due to their amphipathic nature in aqueous media. For instance, hydrophobic compounds entrap in the bilayer membrane, while hydrophilic compounds encapsulate in the aqueous core. Liposomes serve as DDSs due to their versatile structure; biocompatibility; and the fact they are naturally nontoxic, non-immunogenic, and biodegradable. Liposomes have several advantages contributing to drug delivery. They have a role enhancing drug solubility, serving as a sustained release system, providing targeted drug delivery, reducing the toxic effect of drugs, providing protection against drug degradation, enhancing circulation half-life of APIs, being effective in overcoming multidrug resistance, improving the therapeutic index of the entrapped drug, and protecting APIs against their surrounding environment.

Numerous factors define liposomes properties such as the lipid composition, number of lipid bilayers, size, surface charge, and the method of preparation. Liposomal vesicles vary in size between 0.025 μm to 2.5 μm. They can be categorised according to the number of their layers (also referred to as lamellae): unilamellar (consisting of single phospholipid bilayer) or multilamellar (consisting of more than one unilamellar separated by layers of water (>500 nm)). Unilamellar vesicles are subdivided into small unilamellar vesicles (20-100 nm) and large unilamellar vesicles (>100 nm). Both the size and the number of lamellae in the liposomal structure are considered to be the most crucial factors affecting the vesicles half-life and the quantity of API that is to be encapsulated. This unique and flexible variety in the liposomal structure distinguishes liposomes as the preferred carriers for a broad spectrum of therapeutic agents.

Physical and chemical stability of the liposomes in terms of size distribution, entrapment efficiency, and minimal degradation of liposomal apparatuses is the major limiting step for drug delivery using this system. Chemical degradation of liposomes mainly occurs at the phospholipid bilayers level, in which two different reactions might develop: (i) hydrolysis of the ester bonds between fatty acids and glycerol backbone, and (ii) peroxidation of any available unsaturated acyl chain. These two reactions might lead to the development of short-chain lipids; subsequently, soluble derivatives will appear in the membrane that would significantly reduce the quality and stability of the liposomal system. With respect to physical instability, liposomes might undergo aggregation/flocculation and fusion/coalescence, which can ultimately change vesicle size and lead to significant loss of the encapsulated API.

Several factors that have an influence on liposomal system stability, such as liposomal composition (e.g., phospholipids-lipids with high phase transition temperatures), fatty acid side-chains, polar head chemistry, chain length, and the degree of unsaturation, can maintain liposomal rigidity and phospholipid:cholesterol molar ratio (crucial for the liposomal stability and controlling drug release).

Phospholipids are the main building blocks of liposomes. These biomolecules are also the main components building the biological membranes. They are amphiphilic molecules that consist of a polar head (water soluble hydroxy groups) and insoluble backbone. Liposomes can be zwitterionic, positively or negatively charged, or uncharged. This is dependent on the polar head charge. There are two types of lipids currently utilized for liposome preparation: naturally occurring or synthesized double-chain lipids (consisting of phosphorus polar head and glycerol backbone) and sterols (e.g., cholesterol).

The most known lipids used in the liposomal formulations are phosphatidylcholine (zwitterionic), phosphatidylglycerol (negatively charged), phosphatidic acid, phosphatidylethanolamine (zwitterionic), and phosphatidylserine (negatively charged). Positively charged lipids (e.g., N-[1-(2,3-dioleyloxy)propyl]-N,N,N-triethylammonium (DOTMA) and 1,2-dioleoyl-3-trimethylammoniopropane (DOTAP)) are mainly used for gene delivery, as they interact with the negatively charged deoxyribonucleic acid (DNA) and negatively charged APIs.

Cholesterol is another strategic component of liposomes. It has a modulatory effect on the properties of the lipid bilayer of the liposomes. It can control the stoutness in the liposome structure and increase the packing between the phospholipid molecules, resulting in more ordered conformation in the aliphatic tail region, reduced micropolarity, reduced bilayer flexibility to the surrounding molecules (especially water soluble molecules), and increases in the microviscosity of the bilayer. Cholesterol is also crucial for structural stability of liposomal membranes against intestinal environmental stress. Cholesterol was found to influence liposomes size (increasing cholesterol concentration increases liposomes size in addition to shape transition), provide permeability and fluidity, and consequently modulate the release of hydrophilic compounds from liposomes.

The lipid bilayer of a liposome may fuse with other bilayers (e.g., the cell membrane), thus delivering the contents of the liposome to cells. Phospholipids generally comprise two fatty acids linked through glycerol phosphate to one of a variety of polar groups. Non-limiting examples of phospholids suitable for liposomes include phosphatidic acid (PA), phosphatidylserine (PS), phosphatidylinositol (P1), phosphatidylglycerol (PG), diphosphatidylglycerol (DPG), phosphatidylcholine (PC), and phosphatidylethanolamine (PE), or any combination thereof.

The fatty acid chains may range from about 6 to about 26 carbon atoms in length, and the lipid chains may be saturated or unsaturated. Non-limiting examples of suitable fatty acid chains include (common name presented in parentheses) n-dodecanoate (laurate), n-tetradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all-cis-9,12,15-octadecatrienoate (linolenate), and all-cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and any combination thereof.

The phospholipids can come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-diheptyloxacarbocyanine iodide, 1,1′-didodecyl-3,3,3′,3′-tetramethylindocarbocyanine perchloarate, 1,1′-dioleyl-3,3,3′,3′-tetramethylindo carbocyanine methanesulfonate, N,4-(dilinoleylaminostyryl)-N-methylpyridinium iodide, or 1,1-dilinoleyl-3,3,3′,3′-tetramethylindocarbocyanine perchloarate.

Liposomes can optionally comprise sphingolipids, in which spingosine is the structural counterpart of glycerol and one of the one fatty acids of a phosphoglyceride, or cholesterol, a major component of animal cell membranes. Liposomes may optionally, contain pegylated lipids, which are lipids covalently linked to polymers of polyethylene glycol (PEG). The PEGylated lipids may generally increase the amount of compound that can be incorporated into the liposomes. PEGs may range in size from about 500 to about 10,000 Daltons. A suitable PEGylated phospholipid is dipalmitoyl PE bearing PEG 5,000.

Liposomes can further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying the cannabinoids of the instant disclosure can be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, and Verrico et al., PAIN, 2020 Sep. 1; 161(9): 2191-2202, the disclosures of which are hereby incorporated by reference in their entirety. For example, liposomes can be prepared by sonicating lipids in an aqueous solution, solvent injection, lipid hydration, reverse evaporation, or freeze drying by repeated freezing and thawing. The liposomes can be multilamellar, which have many layers like an onion, or unilamellar.

As would be apparent to one of ordinary skill, all the parameters that govern liposome formation may be varied. These parameters include, but are not limited to, temperature, pH, concentration of active pharmaceutical ingredient, concentration and composition of lipid, concentration of multivalent cations, rate of mixing, presence of and concentration of solvent. In some aspects, the liposomes are formed using methods described in the examples herein below.

Liposomes retain the cannabinoid compounds in the lungs longer when compared to cannabinoid compositions that are not encapsulated. Liposomes can localize the action of inhaled cannabinoids in the lung, improving the therapeutic outcome of the medication and reducing systemic adverse effects.

Dipalmitoylphosphatidylcholine liposomes given intratracheally in mice are taken up by pulmonary cells, and more than 50% of the phospholipid administered remained in the lung after 24 hours of administration. Thus, it is also more feasible to reach the ‘deeper’ layers of the bronchio-space by using liposomal vehicles. In some aspects, the liposome comprises 1,2-dilauroyl-sn-glycero-3-phosphocholine (DLPC). In some aspects, when the cannabinoid is CBD, the CBD is mixed with DLPC at a mass ratio of about 1:1, 1:2, 1:3, 1:4, 1:5, 2:1, 3:1, or about 4:1. In some aspects, when the cannabinoid is CBD, the CBD is mixed with DLPC at a mass ratio of about 1:5. In some aspects, the liposomes are formed by sonication/nebulization in PBS. The liposomes can comprise from about 1 to about 40 μg/L, from about 5 to about 30 μg/L, from about 8 to about 25 μg/L, or from about 10 to about 20 μg/ml CBD, about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or about 40 μg/ml. In some aspects, the liposomes comprise from about 10 to about 20 μg/ml CBD.

When the cannabinoid is CBD, the concentration of CBD in the aerosol composition can range from about 1 to about 25 μg/L, from about 5 to about 20 μg/L, from about 10 to about 15 μg/L, or from about 10 to about 13 μg/L, about 1, 2, 3, 4, 5, 6, 7, 8, 9, 10, 11, 12, 13, 14, 15, 16, 17, 18, 19, 20, 21, 22, 23, 24, or about 25 μg/L. In some aspects, the cannabinoid is THC. The THC molecule can be A9-tetrahydrocannabinol, Δ8-tetrahydrocannabinol, 11-hydroxy-tetrahydrocannabinol, 11-hydroxy-Δ9-tetrahydrocannabinol, Δ11-tetrahydrocannabinol, or any combination thereof.

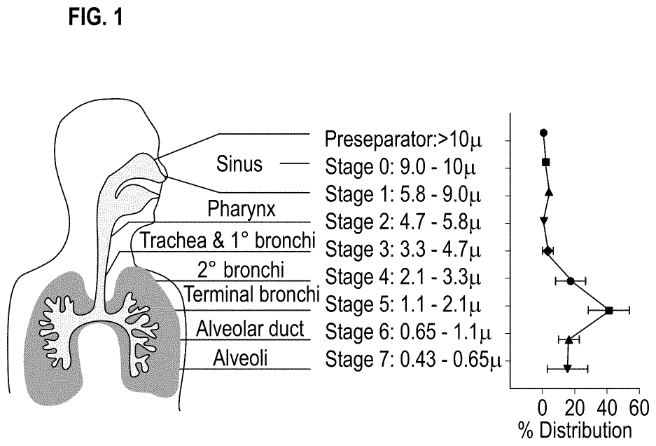

Through extensive experimentation, the inventors discovered that the diameter of liposomes affects efficiency of delivery of the cannabinoids to the various pulmonary spaces. More specifically, the inventors discovered that liposomes having a Mass Mean Aerodynamic Diameter (MMAD; “diameter”) ranging from about 0.1 μm to about 10 μm can deposit an encapsulated cannabinoid in all pulmonary spaces, where the smaller diameter liposomes deposit cannabinoids in the alveoli, and the larger diameter liposomes deposit cannabinoids in the sinuses. FIG. 1 graphically depicts the quantity of CBD as it is found further into the depths of the pulmonary system. The size distribution of a drug delivery system suitable for depositing an encapsulated cannabinoid in all pulmonary spaces can and will vary depending on the drug delivery system, the encapsulated cannabinoid, the target airways in the lung spaces, the flow rate at which the aerosolized composition is delivered to the lungs, and the subject among other variables and can be determined experimentally.

In some aspects, the liposomes of the instant disclosure when used for delivery of cannabinoids to pulmonary airspaces have a diameter of less than about 0.1 μm to more than about 10 μm less than about 0.4 μm to more than about 10 μm or more, less than about 0.4 μm to more than about 9 μm, less than about 0.4 μm to more than about 8 μm, less than about 0.4 μm to more than about 7 μm, less than about 0.4 μm to more than about 6 μm, less than about 0.4 μm to more than about 5 μm, less than about 0.4 μm to more than about 4 μm, less than about 0.4 μm to more than about 3 μm, less than about 0.4 μm to more than about 2 μm, less than about 0.4 μm to more than about 1 μm. In some aspects, the liposomes of the instant disclosure have a diameter of less than about 0.4 μm to more than about 2 μm. In some aspects, the liposomes of the instant disclosure have a diameter of less than about 0.1 μm to more than about 10 μm. In some aspects, the liposomes of the instant disclosure have a diameter of about 0.1 μm to about 10 μm, about 0.4 μm to about 10 μm, about 0.4 μm to about 9 μm, about 0.4 μm to about 8 μm, about 0.4 μm to about 7 μm, about 0.4 μm to about 6 μm, about 0.4 μm to about 5 μm, about 0.4 μm to about 4 μm, about 0.4 μm to about 3 μm, about 0.4 μm to about 2 μm, about 0.4 μm to about 1 μm. In some aspects, the liposomes of the instant disclosure have a diameter of about 0.1 μm to about 10 μm. In some aspects, the liposomes of the instant disclosure have a diameter of about 0.4 μm to about 2 μm. It will be recognized that the diameter of liposomes that can efficiently deliver cannabinoids to the higher and lower pulmonary areas can depend on a number of factors, including the composition of the liposomes and the flow rate at which the aerosolized composition is delivered to the lungs.

In some aspects, when the liposomes are used for delivery of liposomes administered orally, parenterally, or topically, the liposomes have a diameter of less than about 0.01 μm to more than about 10 μm, less than about 0.02 μm to more than about 5 μm or more, less than about 0.03 μm to more than about 1 μm, less than about 0.04 μm to more than about 0.5 μm, less than about 0.05 μm to more than about 0.4 μm, less than about 0.06 μm to more than about 0.3 μm, less than about 0.07 μm to more than about 0.2 μm, less than about 0.08 μm to more than about 0.12 μm, In some aspects, the liposomes of the instant disclosure have a diameter of less than about 0.08 μm to more than about 0.12 μm.

In some aspects, the liposomes of the instant disclosure have a diameter of about 0.01 μm to about 10 μm, about 0.02 μm to about 5 μm or more, about 0.03 μm to about 1 μm, about 0.04 μm to about 0.5 μm, about 0.05 μm to about 0.4 μm, about 0.06 μm to about 0.3 μm, about 0.07 μm to about 0.2 μm, about 0.08 μm to about 0.12 μm. In some aspects, the liposomes of the instant disclosure have a diameter of about 0.08 μm to about 0.12 μm. It will be recognized that the diameter of liposomes that can efficiently deliver cannabinoids to various body parts or tissues can depend on a number of factors, including the composition of the liposomes and the flow rate at which the aerosolized composition is delivered.

(c) Pharmaceutical Compositions

The encapsulated cannabinoid of the instant disclosure can be appropriately formulated in a pharmaceutical composition for delivery of the composition into various body parts or tissues using various routes of administration. The inventors discovered that cannabinoids, when encapsulated, can significantly improve efficacy of administered cannabinoids against inflammation and inflammatory disorders. The pharmaceutical compositions can be formulated by several different means known by individuals of skill in the art. The composition can generally be administered parenterally, intraperitoneally, intravascularly, transdermally, subcutaneously, rectally, or intrapulmonarily such as by inhalation in dosage unit formulations containing conventional nontoxic pharmaceutically acceptable adjuvants, carriers, excipients, and vehicles as desired. The term parenteral as used herein includes subcutaneous, intravenous, intramuscular, intraparenchymal, intrathecal, or intracisternal injection, or infusion techniques. In some aspects, a pharmaceutical composition of the instant disclosure can be formulated for aerosolized administration in the form of a spray, an aerosol, an oil suspension, an emulsion, a solution, and the like in dosage unit formulations containing conventional nontoxic pharmaceutically acceptable adjuvants, excipients, and vehicles as desired. Formulation of pharmaceutical systems is discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

In some aspects, the encapsulated cannabinoid of the instant disclosure are formulated in a pharmaceutical composition for oral administration of the composition. In some aspects, the encapsulated cannabinoid of the instant disclosure are formulated in a pharmaceutical composition for parenteral administration of the composition.

As explained above, the inventors surprisingly discovered that an encapsulated version of cannabinoids of the instant disclosure can be aerosolized to disperse through the lungs more efficiently, reaching higher and lower respiratory spaces, and directly exerting anti-inflammatory activities in lung tissues. Accordingly, in some aspects, the encapsulated cannabinoid of the instant disclosure can be aerosolized in a pharmaceutical composition for delivery of the composition into the upper and lower pulmonary spaces. For instance, a pharmaceutical composition of the instant disclosure can be formulated for aerosolized administration in the form of a spray, an aerosol, an oil suspension, an emulsion, a solution, and the like in dosage unit formulations containing conventional nontoxic pharmaceutically acceptable adjuvants, excipients, and vehicles as desired.

Pharmaceutical compositions of various aspects of the instant disclosure can include the encapsulated cannabinoid of the instant disclosure, along with an excipient and/or pharmaceutically acceptable carrier. Non-limiting examples of excipients include preservatives (antioxidants), flavor-modifying agents, coloring agents, chelating agents, antimicrobial agents, stabilizers, surfactants, tonicity agents such as NaCl, suspending agents, release-controlling polymers, and any combination thereof. As used herein, the term “pharmaceutically acceptable carrier” is intended to include any and all solvents, dispersion media, coatings, antibacterial and antifungal agents, isotonic and absorption delaying agents, and other excipients compatible with pharmaceutical administration. The use of such media and agents for pharmaceutically active substances is well known in the art. It will be recognized that carriers can provide more than one function in a composition. A more detailed description of carriers can be as described herein below.

i. Preservatives

Non-limiting examples of preservatives include, but are not limited to, ascorbic acid and its salts, ascorbyl palmitate, ascorbyl stearate, anoxomer, N-acetylcysteine, benzyl isothiocyanate, m-aminobenzoic acid, o-aminobenzoic acid, p-aminobenzoic acid (PABA), butylated hydroxyanisole (BHA), butylated hydroxytoluene (BHT), caffeic acid, canthaxantin, alpha-carotene, beta-carotene, beta-caraotene, beta-apocarotenoic acid, carnosol, carvacrol, catechins, cetyl gallate, chlorogenic acid, citric acid and its salts, clove extract, coffee bean extract, p-coumaric acid, 3,4-dihydroxybenzoic acid, N,N′-diphenyl-p-phenylenediamine (DPPD), dilauryl thiodipropionate, distearyl thiodipropionate, 2,6-di-tert-butylphenol, dodecyl gallate, edetic acid, ellagic acid, erythorbic acid, sodium erythorbate, esculetin, esculin, 6-ethoxy-1,2-dihydro-2,2,4-trimethylquinoline, ethyl gallate, ethyl maltol, ethylenediaminetetraacetic acid (EDTA), eucalyptus extract, eugenol, ferulic acid, flavonoids (e.g., catechin, epicatechin, epicatechin gallate, epigallocatechin (EGC), epigallocatechin gallate (EGCG), polyphenol epigallocatechin-3-gallate), flavones (e.g., apigenin, chrysin, luteolin), flavonols (e.g., datiscetin, myricetin, daemfero), flavanones, fraxetin, fumaric acid, gallic acid, gentian extract, gluconic acid, glycine, gum guaiacum, hesperetin, alpha-hydroxybenzyl phosphinic acid, hydroxycinammic acid, hydroxyglutaric acid, hydroquinone, N-hydroxysuccinic acid, hydroxytryrosol, hydroxyurea, rice bran extract, lactic acid and its salts, lecithin, lecithin citrate; R-alpha-lipoic acid, lutein, lycopene, malic acid, maltol, 5-methoxy tryptamine, methyl gallate, monoglyceride citrate; monoisopropyl citrate; morin, beta-naphthoflavone, nordihydroguaiaretic acid (NDGA), octyl gallate, oxalic acid, palmityl citrate, phenothiazine, phosphatidylcholine, phosphoric acid, phosphates, phytic acid, phytylubichromel, pimento extract, propyl gallate, polyphosphates, quercetin, trans-resveratrol, rosemary extract, rosmarinic acid, sage extract, sesamol, silymarin, sinapic acid, succinic acid, stearyl citrate, syringic acid, tartaric acid, thymol, tocopherols (i.e., alpha-, beta-, gamma- and delta-tocopherol), tocotrienols (i.e., alpha-, beta-, gamma- and delta-tocotrienols), tyrosol, vanillic acid, 2,6-di-tert-butyl-4-hydroxymethylphenol (i.e., lonox 100), 2,4-(tris-3′,5′-bi-tert-butyl-4′-hydroxybenzyl)-mesitylene (i.e., lonox 330), 2,4,5-trihydroxybutyrophenone, ubiquinone, tertiary butyl hydroquinone (TBHQ), thiodipropionic acid, trihydroxy butyrophenone, tryptamine, tyramine, uric acid, vitamin K and derivatives, vitamin Q10, wheat germ oil, zeaxanthin, or combinations thereof.

ii. Chelating Agents

A chelating agent may be included as an excipient to immobilize oxidative groups, including but not limited to metal ions, in order to inhibit the oxidative degradation of the morphinan by these oxidative groups. Non-limiting examples of chelating agents include lysine, methionine, glycine, gluconate, polysaccharides, glutamate, aspartate, and disodium ethylenediaminetetraacetate (Na2EDTA).

iii. Flavor-Modifying Agents

Suitable flavor-modifying agents include flavorants, taste-masking agents, sweeteners, and the like. Flavorants include, but are not limited to, synthetic flavor oils and flavoring aromatics and/or natural oils, extracts from plants, leaves, flowers, fruits, and combinations thereof. Other non-limiting examples of flavors include cinnamon oils, oil of wintergreen, peppermint oils, clover oil, hay oil, anise oil, eucalyptus, vanilla, citrus oils such as lemon oil, orange oil, grape and grapefruit oil, fruit essences including apple, peach, pear, strawberry, raspberry, cherry, plum, pineapple, and apricot.

Taste-masking agents include, but are not limited to, cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other aspects, additional taste-masking agents contemplated are those described in U.S. Pat. Nos. 4,851,226; 5,075,114; and 5,876,759, each of which is hereby incorporated by reference in its entirety.

Non-limiting examples of sweeteners include glucose (corn syrup), dextrose, invert sugar, fructose, and mixtures thereof (when not used as a carrier); saccharin and its various salts such as the sodium salt; dipeptide sweeteners such as aspartame; dihydrochalcone compounds, glycyrrhizin; Stevia rebaudiana (Stevioside); chloro derivatives of sucrose such as sucralose; sugar alcohols such as sorbitol, mannitol, sylitol, hydrogenated starch hydrolysates and the synthetic sweetener 3,6-dihydro-6-methyl-1,2,3-oxathiazin-4-one-2,2-dioxide, particularly the potassium salt (acesulfame-K), and sodium and calcium salts thereof.

iv. Colorants

Depending upon the aspect, it may be desirable to include a coloring agent. Suitable color additives include, but are not limited to, food, drug and cosmetic colors (FD&C), drug and cosmetic colors (D&C), or external drug and cosmetic colors (Ext. D&C). These colors or dyes, along with their corresponding lakes, and certain natural and derived colorants, may be suitable for use in various aspects.

v. Chelating Agents

A chelating agent may be included as an excipient to immobilize oxidative groups, including but not limited to metal ions, in order to inhibit the oxidative degradation of the morphinan by these oxidative groups. Non-limiting examples of chelating agents include lysine, methionine, glycine, gluconate, polysaccharides, glutamate, aspartate, and disodium ethylenediaminetetraacetate (Na2EDTA).

vi. Antimicrobial Agents

An antimicrobial agent may be included as an excipient to minimize the degradation of the compound according to this disclosure by microbial agents, including but not limited to, bacteria and fungi. Non-limiting examples of antimicrobials include para-bens, chlorobutanol, phenol, calcium propionate, sodium nitrate, Na2EDTA, and sulfites, including but not limited to sulfur dioxide, sodium bisulfite, and potassium hydrogen sulfite.

vii. Stabilizers

In some aspects, isotonifiers, sometimes known as “stabilizers”, are added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive, which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers such as polyvinylpyrrolidone monosaccharides, such as xylose, mannose, fructose, glucose; disaccharides such as lactose, maltose, sucrose and trisaccacharides such as raffinose; and polysaccharides such as dextran. In some aspects, the composition does not include stabilizers.

viii. Antimicrobial Agents

An antimicrobial agent may be included as an excipient to minimize the degradation of the compound according to this disclosure by microbial agents, including but not limited to, bacteria and fungi. Non-limiting examples of antimicrobials include para-bens, chlorobutanol, phenol, calcium propionate, sodium nitrate, Na2EDTA, and sulfites, including but not limited to sulfur dioxide, sodium bisulfite, and potassium hydrogen sulfite.

ix. Stabilizers

In some aspects, isotonifiers, sometimes known as “stabilizers”, are added to ensure isotonicity of liquid compositions of the present disclosure and include polyhydric sugar alcohols, for example trihydric or higher sugar alcohols, such as glycerin, erythritol, arabitol, xylitol, sorbitol and mannitol. Stabilizers refer to a broad category of excipients which can range in function from a bulking agent to an additive, which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers such as polyvinylpyrrolidone monosaccharides, such as xylose, mannose, fructose, glucose; disaccharides such as lactose, maltose, sucrose and trisaccacharides such as raffinose; and polysaccharides such as dextran.

x. Surfactants

Non-ionic surfactants or detergents (also known as “wetting agents”) can be used to help solubilize the therapeutic agent, as well as to protect the active ingredient against agitation-induced aggregation. Suitable non-ionic surfactants include polysorbates (20, 80, etc.), polyoxamers (184, 188, etc.), Pluronic polyols, and polyoxyethylene sorbitan monoethers (TWEEN®-20, TWEEN®-80, etc.).

### II. Method of Treating

Another aspect of the present disclosure encompasses a method of providing a protective effect, therapeutic effect, or any combination thereof from inflammation or an inflammatory disorder to a subject in need thereof. See for example, Verrico et al., 2020, Pain. 2020 Sep. 1; 161(9): 2191-2202, the disclosure of all of which is incorporated herein in its entirety. Providing a protective effect can prevent, delay, or arrest development of the disease condition in the subject. The method comprises administering a therapeutically effective amount of a pharmaceutical composition comprising an encapsulated cannabinoid. In some aspects, a method of the instant disclosure comprises treating pulmonary inflammation and disorders associated with inflammation in a subject in need thereof. The method comprises administering to the pulmonary spaces of a subject a therapeutically effective amount of a composition comprising an encapsulated cannabinoid. The method can further comprise aerosolizing the pharmaceutical composition before administration by inhalation. Cannabinoids, encapsulated cannabinoids, and pharmaceutical compositions comprising the encapsulated cannabinoids can be as described in Section I herein above.

The subject can be a human, a livestock animal, a companion animal, a lab animal, or a zoological animal. In one aspect, the subject may be a rodent, e.g., a mouse, a rat, a guinea pig, etc. Non-limiting examples of suitable livestock animals may include pigs, cows, horses, goats, sheep, llamas and alpacas. Non-limiting examples of companion animals may include pets such as dogs, cats, rabbits, and birds. As used herein, a “zoological animal” refers to an animal that may be found in a zoo. Such animals may include non-human primates, large cats, wolves, and bears. Non-limiting examples of a laboratory animal may include rodents, canines, felines, and non-human primates. Non-limiting examples of rodents may include mice, rats, guinea pigs, etc. In some aspects, the subject is a human subject.

(a) Inflammation

In some aspects, the disease condition is inflammation or a condition associated with inflammation. Inflammation can be in any cell type of any tissue or organ. For instance, inflammation in the eye can be in rod photoreceptor cells, cone photoreceptor cells, retinal pigment epithelial (RPE) cells, or other. Non-limiting examples of disorders associated with, caused by, or resulting from inflammation include Inflammatory disorders include encephalitis, myelitis, meningitis; arachnoiditis; neuritis; dacryoadenitis; scleritis; episcleritis; keratitis; retinitis; chorioretinitis; blepharitis; conjunctivitis; uveitis; otitis externa; otitis media; labyrinthitis; mastoiditis; carditis; endocarditis; myocarditis; pericarditis; vasculitis; arteritis; phlebitis; capillaritis; sinusitis; rhinitis; allergic rhinitis, pharyngitis; laryngitis; tracheitis; bronchitis; pneumonitis; pleuritis; mediastinitis; stomatitis; gingivitis; gingivostomatitis; glossitis; tonsillitis; sialadenitis/parotitis; cheilitis; pulpitis; gnathitis; esophagitis; gastritis; gastroenteritis; enteritis; gastrointestinal conditions such as inflammatory bowel disease, Crohn's disease, gastritis, irritable bowel syndrome, or ulcerative colitis; colitis; enterocolitis; duodenitis; ileitis; caecitis; appendicitis; proctitis; hepatitis; ascending cholangitis; cholecystitis; pancreatitis; peritonitis; folliculitis; cellulitis; hidradenitis; dermatomyositis; myositis; synovitis/tenosynovitis; enthesitis; fasciitis; capsulitis; epicondylitis; tendinitis; panniculitis; osteochondritis: osteitis/osteomyelitis; spondylitis; periostitis; chondritis; nephritis; glomerulonephritis; pyelonephritis; ureteritis; cystitis; urethritis; oophoritis; salpingitis; endometritis; parametritis; cervicitis; vaginitis; vulvitis; mastitis; orchitis; epididymitis; prostatitis; seminal vesiculitis; balanitis; posthitis; balanoposthitis; chorioamnionitis; funisitis; omphalitis; insulitis; hypophysitis; thyroiditis; parathyroiditis; adrenalitis; lymphangitis; lymphadenitis, arthritic conditions including, but not limited to, rheumatoid arthritis, spondyloarthropathies, gouty arthritis, osteoarthritis, systemic lupus erythematosus, or juvenile arthritis, asthma, bronchitis, menstrual cramps, premature labor, bursitis, skin-related conditions such as psoriasis, eczema, burns, dermatitis, vascular diseases such as migraine headaches, periarteritis nodosa, aplastic anemia, Hodgkin's disease, scleroderma, rheumatic fever, type I diabetes, neuromuscular junction disease including myasthenia gravis, white matter disease, multiple sclerosis, sarcoidosis, nephrotic syndrome, Behcet's syndrome, polymyositis, hypersensitivity, respiratory distress syndrome, endotoxin shock syndrome, atherosclerosis, cancers, conditions associated with pulmonary inflammation, such as that associated with viral infections or cystic fibrosis, neuroinflammatory diseases such as Alzheimer's disease (AD), stroke, traumatic brain injury (TBI), Parkinson's disease (PD), Amyotrophic lateral sclerosis (ALS), multiple sclerosis (MS), ataxia, Bell's palsy, or epilepsy.

In some aspects, pharmaceutical compositions comprise unit dose forms comprising an amount of cannabinoids per dose. In some aspects, the inflammatory disorder is non-eosinophilic inflammation or inflammatory disorder and the method comprises treating the non-eosinophilic inflammation or inflammatory disorder by administering a unit dose of the pharmaceutical composition to a subject in need thereof. In some aspects, the encapsulated cannabinoid is encapsulated CBD, and the method comprises administering a unit dose of a pharmaceutical composition comprising encapsulated CBD to a subject in need thereof.

The concentration of CBD in a unit doe can and will vary depending on the inflammation or inflammatory disorder, the route of administration, and the subject among other variables. A unit dose form of the composition can comprise about 1, 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or about 200 mg of CBD or more, from about 10 to about 200 mg of CBD, from about 20 to about 190 mg of CBD, from about 30 to about 180 mg of CBD, from about 40 to about 170 mg of CBD, from about 50 to about 160 mg of CBD, from about 60 to about 150 mg of CBD, from about 70 to about 140 mg of CBD, from about 80 to about 130 mg of CBD, from about 90 to about 120 mg of CBD, from about 90 to about 110 mg of CBD, or from about 195 to about 105 mg of CBD. In some aspects, the composition is formulated for oral administration to a human subject and a unit dose form of the composition comprises from about 90 to about 110 mg of CBD. In some aspects, the composition is formulated for oral administration to a human subject and a unit dose form of the composition comprises from about 95 to about 105 mg of CBD.

In some aspects, the inflammatory disorder is osteoarthritis. When the inflammatory disorder is osteoarthritis, the method can comprise orally administering to a human subject about 50 to about 160 mg of CBD, from about 60 to about 150 mg of CBD, from about 70 to about 140 mg of CBD, from about 80 to about 130 mg of CBD, from about 90 to about 120 mg of CBD, from about 90 to about 110 mg of CBD, or from about 195 to about 105 mg of CBD. In some aspects, the composition is formulated for oral administration to a human subject and the method comprises administering from about 90 to about 110 mg of CBD. In some aspects, the inflammatory disorder is osteoarthritis, and the method comprises orally administering to a human subject from about 90 to about 110 mg of CBD. In some aspects, the inflammatory disorder is osteoarthritis, and the method comprises orally administering to a human subject from about 95 to about 105 mg of CBD.

When the inflammatory disorder is osteoarthritis, the method can comprise orally administering to a human subject about 1 to about 40 mg/kg of CBD, from about 2 to about 39 mg of CBD, from about 3 to about 38 mg of CBD, from about 4 to about 37 mg of CBD, from about 5 to about 36 mg of CBD, from about 6 to about 35 mg of CBD, from about 7 to about 34 mg of CBD, from about 8 to about 33 mg of CBD, from about 9 to about 32 mg of CBD, from about 10 to about 31 mg of CBD, from about 11 to about 30 mg of CBD, from about 12 to about 29 mg of CBD, from about 13 to about 28 mg of CBD, from about 14 to about 27 mg of CBD, from about 15 to about 26 mg of CBD, from about 16 to about 25 mg of CBD, from about 17 to about 24 mg of CBD, from about 18 to about 23 mg of CBD, from about 19 to about 22 mg of CBD, or from about 19 to about 21 mg of CBD. In some aspects, the composition is formulated for oral administration to a human subject and the method comprises administering from about 18 to about 22 mg of CBD. In some aspects, the inflammatory disorder is osteoarthritis, and the method comprises orally administering to a human subject from about 18 to about 22 mg of CBD. In some aspects, the inflammatory disorder is osteoarthritis, and the method comprises orally administering to a human subject from about 19 to about 21 mg of CBD.

A. Acute Inflammation

In some aspects, a method of the instant disclosure comprises treating an acute inflammation. Acute inflammation occurs immediately upon injury, lasting only a few days. Cytokines and chemokines promote the migration of neutrophils and macrophages to the site of inflammation. Pathogens, allergens, toxins, burns, and frostbite are some of the typical causes of acute inflammation. Acute inflammation can be a defensive mechanism to protect tissues against injury. Inflammation lasting 2-6 weeks is designated subacute inflammation. Acute inflammation may be regarded as the first line of defense against injury. Acute inflammatory response requires constant stimulation to be sustained. Inflammatory mediators are short-lived and are quickly degraded in the tissue. Hence, acute inflammation begins to cease once the stimulus has been removed. Acute inflammation can be categorized as mild, moderate, severe, and systemic severe inflammation. Symptoms of mild, moderate, severe, and systemic severe inflammation can and will vary depending on the type of inflammation and can be as recognized in the art for each type of inflammation. For instance, a mild viral infection in the lungs can develop into severe inflammation during emphysema.

Acute inflammation is a short-term process, usually appearing within a few minutes or hours and begins to cease upon the removal of the injurious stimulus. It involves a coordinated and systemic mobilization response locally of various immune, endocrine, and neurological mediators of acute inflammation. In a normal healthy response, it becomes activated, clears the pathogen or source of injury, begins a repair process, and then ceases. It is characterized by five cardinal signs: pain, calor heat, redness, swelling, and loss of function. Redness and heat are due to increased blood flow at body core temperature to the inflamed site; swelling is caused by accumulation of fluid; and pain is due to the release of chemicals such as bradykinin and histamine that stimulate nerve endings. Loss of function has multiple causes. Swelling in the lungs caused by acute inflammation can lead to shortness of breath, cough, fatigue, loss of appetite, and unintentional weight loss.

An infectious organism can escape the confines of the immediate tissue via the circulatory system or lymphatic system, where it may spread to other parts of the body to cause severe acute inflammation. If an organism is not contained by the actions of acute inflammation, it may gain access to the lymphatic system via nearby lymph vessels. An infection of the lymph vessels is known as lymphangitis, and infection of a lymph node is known as lymphadenitis. When lymph nodes cannot destroy all pathogens, the infection spreads further. A pathogen can gain access to the bloodstream through lymphatic drainage into the circulatory system. When inflammation overwhelms the host, systemic inflammatory response syndrome is diagnosed. When it is due to infection, the term sepsis is applied, with the term's bacteremia being applied specifically for bacterial sepsis and viremia specifically to viral sepsis as is the case with COVID-19. Vasodilation and organ dysfunction are serious problems associated with widespread infection that may lead to septic shock and death.

B. Chronic Inflammation

In some aspects, a method of the instant disclosure comprises treating chronic inflammation or inflammatory disorders. Chronic inflammation is the result of release of pro-inflammatory cytokines from immune-related cells and the chronic activation of the innate immune system. Chronic inflammation can last for months or years. Neutrophils are beneficial against invading pathogens in the host, but exert adverse effects in a number of diseases including those of the lung. Chronic pulmonary inflammation can contribute to the development or progression of certain conditions such as chronic obstructive pulmonary disease (COPD), cystic fibrosis (CF), acute lung injury (ALI), and asthma. Obesity, smoking, stress and insufficient diet are some of the factors that promote chronic inflammation. Common signs and symptoms that develop during chronic inflammation include body pain, arthralgia, myalgia, chronic fatigue and insomnia, depression, anxiety, and mood disorders, gastrointestinal complications such as constipation, diarrhea, and acid reflux, weight gain or loss, and frequent infections.

(b) Pulmonary Inflammation

In some aspect, the method comprises treating a pulmonary inflammation or inflammatory disorder. Lung inflammation can be characterized by an increase in active WBCs compared to WBC's of a healthy subject without lung inflammation. Several inflammatory situations in the airways are observed during chronic infections and are a result of accumulated and activated neutrophils, activated cells from the monocytic lineage, over-activated T cells, and/or a highly concentrated milieu of inflammatory cytokines, including but not limited to, TNFa, IL-6, IL-18, IL-4, IL-5, IL-13, IL-2, INFg, IL-1 a, IL-1b, IL-23, IL-17, IL-8, or any combination thereof. Additionally, several of these can become quickly and dangerously elevated during acute infections and may be equally treatable as mentioned herein.

Neutrophils, the most abundant cell type in the blood in humans, are a fundamental component of the innate immune response. It is estimated that each day 1 billion neutrophils are produced per kilogram of body weight and that this can increase to 10 billion during an infection. At steady state, developing neutrophils reside in the bone marrow, while mature neutrophils are released into the circulation and rapidly recruited into affected tissues in response to infection or injury. Neutrophils are short-lived, although their precise life span is debated. As the most abundant and short-lived cell in the circulation, neutrophil turnover must be tightly regulated during both homeostasis and disease.

Although neutrophils are critical to the immune system in the event of microbial infections, an overabundance of neutrophils (neutrophilia) in circulation or in tissues has been implicated to be a problem in several lung diseases. Airway neutrophilia is an increased neutrophilic infiltration in the lungs and is implicated in pathogenic processes leading to the tissue damage associated with many acute and chronic lung diseases and conditions. A massive influx of neutrophils is seen in acute pulmonary infections, pneumonia, and sepsis. Many of these lung conditions lead to acute lung injury (ALI) and tissue damage. Neutrophils employ several pro-inflammatory mechanisms, including the release of pro-inflammatory cytokines such as TNFα and IL-113, cytokines that have been associated with SA. Neutrophils and neutrophil extracellular traps (NETs) found in these inflammatory conditions cause tissue injury and severe inflammation in the lung. NETs are extracellular DNA complexed with antimicrobial proteins and help to fight infectious agents. However, an excess of NETs contributes to the pathology of a number of diseases. In the lungs, NETs have been identified in conditions of CF, ALI, and infections with bacteria, fungi, and viruses.

Non-limiting examples of diseases or conditions associated with neutrophilic inflammation include acute respiratory distress syndrome (ARDS), asthma, chronic obstructive pulmonary disease (COPD), allergic bronchopulmonary aspergillosis, hypersensitivity pneumonia, bronchiectasis, emphysema, bronchitis, allergic bronchitis bronchiectasis, cystic fibrosis, tuberculosis, hypersensitivity pneumonitis, occupational asthma, acute lung injury (ALI), sarcoid, reactive airway disease syndrome, interstitial lung disease, rhinitis, sinusitis, exercise-induced asthma, pollution-induced asthma, cough variant asthma, parasitic lung disease, lung cancer, bacterial infections, respiratory syncytial virus (RSV) infection, parainfluenza virus (PIV) infection, rhinovirus (RV) infection and adenovirus infection among others.

As explained in Section I herein above, the inventors discovered that cannabinoids can selectively treat pulmonary neutrophilic inflammation and pulmonary disorder associated with inflammation (inflammatory disorder) but not eosinophilic disorders. Further, the inventors discovered that cannabinoids can even exacerbate pulmonary eosinophilic inflammation and inflammatory disorders. Accordingly, a method of the instant disclosure comprises selectively treating pulmonary inflammation and disorders associated with inflammation caused by or exacerbated by neutrophils. A method of the instant disclosure also comprises selectively treating non-eosinophilic pulmonary inflammation and disorders. Further, a method of the instant disclosure comprises preventing exacerbation of a pulmonary inflammation or disorders associated with inflammation caused by or exacerbated by eosinophils.

In some aspects, a method of the instant disclosure comprises treating a neutrophilic pulmonary inflammation or inflammatory disorder in a subject in need thereof by administering to the subject a therapeutically effective amount of a pharmaceutical composition comprising an encapsulated cannabinoid of the instant disclosure. In other aspects, a method of the instant disclosure comprises treating a non-eosinophilic pulmonary inflammation or inflammatory disorder by administering to the subject a therapeutically effective amount of a pharmaceutical composition comprising an encapsulated cannabinoid of the instant disclosure. In yet other aspects, a method of the instant disclosure comprises treating a steroid-resistant pulmonary inflammation or inflammatory disorder by administering to the subject a therapeutically effective amount of a pharmaceutical composition comprising an encapsulated cannabinoid of the instant disclosure. The method can further comprise diagnosing or having diagnosed non-eosinophilic inflammation, neutrophilic inflammation, or steroid-resistant inflammation before administering to the subject a therapeutically effective amount of the pharmaceutical composition comprising an encapsulated cannabinoid of the instant disclosure. The method can reduce inflammation and/or control, reduce the severity of, or eliminate symptoms of pulmonary neutrophilic inflammation or inflammatory disorders by about 1%, 10%, 20%, 30%, 40%, 50%, 60%, 70%, 80%, 90%, or eliminate the pulmonary neutrophilic inflammation or inflammatory disorder.

In some aspects, a method of the instant disclosure comprises preventing exacerbation of a pulmonary inflammation or inflammatory disorder caused by or exacerbated by eosinophils in a subject in need thereof. The method comprises preventing administration of a pharmaceutical composition comprising an encapsulated cannabinoid of the instant disclosure to the subject. For instance, preventing administration of the pharmaceutical composition of the instant disclosure can comprise noting on a medical chart of the subject a warning against administration of the pharmaceutical composition of the instant disclosure. The method can further comprise diagnosing or having diagnosed non-eosinophilic inflammation, neutrophilic inflammation, or steroid-resistant inflammation before preventing administration of the therapeutically effective amount of the pharmaceutical composition comprising an encapsulated cannabinoid of the instant disclosure to the subject.

A method of the instant disclosure can treat severe asthma (SA). About 25 million Americans (and about 300 million humans) suffer from asthma. It is reported that between 10%-25% are steroid resistant and do not respond to glucocorticoids (GCs) therapy. Steroid-resistant asthma can often lead to death of the patient for lack of treatment options. Within this population it has been observed that polymorphonuclear neutrophil (PMN) accumulation and activity often predominates the cellular makeup of the diseased airway space. Steroid-unresponsive uncontrolled asthma is a hallmark of severe asthma (SA), and an ongoing impediment in the development of new therapies for SA has been a limited understanding of the range of immune responses and molecular networks that contribute to the disease process. This chronically diseased population accounts for more than 50% of direct and indirect healthcare costs associated with asthma. The percentage of neutrophils in the sputum of patients with SA is ≥40% higher than those with mild to moderate asthma. Importantly, the extent of neutrophilic inflammation has been shown to be positively associated with the severity of the disease and steroid-unresponsiveness in SA.

In some aspects, a method of the instant disclosure comprises treating non-eosinophilic asthma. In other aspects, a method of the instant disclosure comprises treating severe asthma (SA). In some aspects, a method of the instant disclosure comprises treating neutrophilic asthma. In yet other aspects, a method of the instant disclosure comprises treating steroid-resistant asthma.

In some aspects, the method comprises treating non-eosinophilic asthma by administering a therapeutically effective amount of the cannabinoid to a subject in need thereof. In some aspects, the method comprises treating severe asthma by administering a therapeutically effective amount of the cannabinoid to a subject in need thereof. In some aspects, the method comprises treating neutrophilic asthma by administering a therapeutically effective amount of the cannabinoid to a subject in need thereof. In some aspects, the encapsulated cannabinoid is encapsulated CBD, and the method comprises treating non-eosinophilic, severe, or neutrophilic asthma by administering a therapeutically effective amount of a pharmaceutical composition comprising encapsulated CBD to a subject in need thereof. The method can control, reduce the severity of, or eliminate symptoms of SA by about 1%, 10%, 20%, 30%, 40%, 50%, 60%, 70%, 80%, 90% or eliminate inflammation or an inflammatory disorder.

In some aspects, a method of the instant disclosure comprises preventing exacerbation of asthma caused by or exacerbated by eosinophils in a subject in need thereof. In some aspects, the encapsulated cannabinoid is encapsulated CBD, and the method comprises preventing exacerbation of asthma caused by or exacerbated by eosinophils by preventing administration of a therapeutically effective amount of a pharmaceutical composition comprising encapsulated CBD to a subject in need thereof. The method can control, reduce the severity of, or eliminate symptoms of SA by about 1%, 10%, 20%, 30%, 40%, 50%, 60%, 70%, 80%, 90% or eliminate inflammation or an inflammatory disorder.

In some aspects, a method of the instant disclosure comprises preventing exacerbation of eosinophilic asthma. Eosinophilic asthma (EA) is marked by high levels of white eosinophils. Eosinophils fight infections and attack invading bacteria. However, in people with EA, eosinophils cause inflammation and swelling in the airways and respiratory system. The higher the levels of eosinophils in the blood, the more severe the symptoms of asthma can be. Unlike neutrophilic asthma, EA is rare. It's not clear how many people have this subtype of asthma, but researchers believe about 5 percent of all adults with asthma have EA.

Administration of encapsulated cannabinoids of the instant disclosure can control, reduce the severity, or eliminate symptoms of asthma, functional limitations, impairment in quality of life, and risk of adverse events that are associated with the asthma. Severity can be defined as the intrinsic intensity of the disease process. Severity is most easily and directly measured in a patient who is not currently receiving long-term control treatment. Control can be defined as the degree to which the manifestations of asthma (symptoms, functional impairments, and risks of untoward events) are minimized and the goals of therapy are met. Methods of assessing the presence and severity of asthma in a subject are known in the art and include physical examination, including hyperexpansion of the thorax, sounds of wheezing during normal breathing, or a prolonged phase of forced exhalation, increased nasal secretion, mucosal swelling, and/or nasal polyps, and atopic dermatitis/eczema or any other manifestation of an allergic skin condition; pulmonary function testing such as spirometry measurements, measurement of lung volumes and evaluation of inspiratory loops, diffusing capacity testing, bronchoprovocation, chest x ray, allergy testing, and use of biomarkers of inflammation.

In some aspects, a method of the instant disclosure is used to treat severe cystic fibrosis (CF). CF is a disease characterized by chronic inflammation and immunemediated damage to the lung and airway, resulting in respiratory failure and death. Neutrophils play a role in CF.

In some aspects, a method of the instant disclosure is used to treat COPD. COPD, or chronic obstructive pulmonary disease, is a progressive disease that makes it hard to breathe. COPD can cause coughing that produces large amounts of mucus (a slimy substance), wheezing, shortness of breath, chest tightness, and other symptoms. In COPD, less air flows in and out of the airways because of one or more of the following: the airways and air sacs lose their elastic quality; the walls between many of the air sacs are destroyed; the walls of the airways become thick and inflamed, and the airways make more mucus than usual, which can clog them. Eosinophils contribute to inflammation that promotes airway obstruction in COPD.

In some aspects, a method of the instant disclosure is used to treat chronic bronchitis. In chronic bronchitis, the lining of the airways is constantly irritated and inflamed. This causes the lining to thicken. Lots of thick mucus forms in the airways, making it hard to breathe.

In some aspects, a method of the instant disclosure is used to treat lung cancer. Lung cancer, also known as lung carcinoma (since about 98-99% of all lung cancers are carcinomas) derives from transformed, malignant cells that originate as epithelial cells, or from tissues composed of epithelial cells. Other lung cancers, such as the rare sarcomas of the lung, are generated by the malignant transformation of connective tissues, which arise from mesenchymal cells. Lymphomas and melanomas (from lymphoid and melanocyte cell lineages) can also rarely result in lung cancer. The two main types are small-cell lung carcinoma (SCLC) and non-small-cell lung carcinoma (NSCLC). The most common symptoms are coughing (including coughing up blood), weight loss, shortness of breath, and chest pains. At least in NSCLC, neutrophils dominate the immune cell composition.

Methods of administering a composition of the instant disclosure to pulmonary spaces for treating a pulmonary inflammation or inflammatory disorder can be as described in Section III herein below.

### III. Aerosolized Administration

As described in Section I(c) herein above, the inventors surprisingly discovered that encapsulating cannabinoids and administering the encapsulated cannabinoids by inhalation of aerosolized encapsulated cannabinoids can deliver therapeutically effective amounts of the cannabinoids directly into all pulmonary spaces to effectively treat pulmonary inflammation and inflammatory disorders, all while avoiding the toxicity and other disadvantages associated with currently available inhalation and systemic delivery modalities of cannabinoids. The Accordingly, one aspect of the present disclosure encompasses a method of administering a therapeutically effective amount of a cannabinoid to pulmonary spaces of a subject in need thereof. The method comprises administering a pharmaceutical composition comprising an encapsulated cannabinoid to the subject by inhalation. The cannabinoid is encapsulated in a drug delivery system comprising a heterogeneous distribution of diameters. The method can further comprise aerosolizing the pharmaceutical composition before administering the aerosolized pharmaceutical composition to the subject by inhalation. Advantageously, the method can deliver the encapsulated cannabinoid to all spaces in the lungs, thereby significantly improving efficacy of the cannabinoid in treating pulmonary inflammation and inflammatory disorders. Cannabinoids, encapsulated cannabinoids, and pharmaceutical compositions comprising the encapsulated cannabinoids can be as described in Section I herein above.

The encapsulated cannabinoid compositions of the instant disclosure can be aerosolized for administration by inhalation. The compositions of the present disclosure can be delivered via any aerosolization methods known to those skilled in the art. Such aerosolization methods and devices include, but are not limited to, metered dose inhalers with propellants such as CFC or HFA or propellants that are physiologically and environmentally acceptable. Other included devices are breath-operated inhalers, multidose dry powder inhalers and aerosol nebulizers. One preferred way of administering the formulations of the invention is by using conventional actuators. The term “actuator” as used in the present invention includes all types of actuators presently available including but not limited to standard metered dose inhalers or breath operated inhalers. Breath-actuated devices are also known, and have been the subject of many patent applications. Thus, for example, GB 1288971; GB 1297993; GB 1335378; GB 1383761; GB 1392192; GB 1413285; WO85/01880; GB 2204799; U.S. Pat. No. 4,803,978 and EP 0186280A describe inhalation-actuated dispensing devices for use with a pressurized aerosol-dispensing container.

In some aspects, administration is effected by a means of a pump or squeeze-actuated nebulizer. In some aspects, the nebulizer comprises a flow rate ranging from about 5 to about 15 L/min or about 10 L/min. In some aspects the nebulizer is Aerotech II comprising a reservoir having a volume of about 1 mg/mL. In some aspects, the dose is delivered in about 30 min.

Actual dosage levels of active ingredients in a therapeutic composition of the disclosure may be varied so as to administer an amount of cannabinoids that is effective to achieve the desired therapeutic response for a particular subject. A selected dosage level may depend upon a variety of factors, including the cannabinoid in a composition, the activity of the therapeutic composition, formulation, the combination with other drugs or treatments, disease and longevity, the pulmonary inflammation and inflammatory disorder, and the physical condition and prior medical history of the subject being treated. Determination of the proper dosage for a particular situation is within the skill of the practitioner.

In some aspects, aerosolizable pharmaceutical compositions comprise unit dose forms comprising an amount of cannabinoids per dose. In some aspects, the method comprises aerosolizing a pharmaceutical composition comprising an encapsulated cannabinoid and administering a unit dose of the aerosolized pharmaceutical composition to a subject in need thereof by inhalation. In some aspects, the inflammatory disorder is non-eosinophilic asthma and the method comprises treating non-eosinophilic asthma by aerosolizing a pharmaceutical composition comprising an encapsulated cannabinoid and administering a unit dose of the aerosolized pharmaceutical composition to a subject in need thereof by inhalation. In some aspects, the encapsulated cannabinoid is encapsulated CBD, and the method comprises aerosolizing a pharmaceutical composition comprising encapsulated CBD and administering a unit dose of the aerosolized pharmaceutical composition to a subject in need thereof by inhalation. In some aspects, the encapsulated cannabinoid is encapsulated CBD, the inflammatory disorder is non-eosinophilic asthma, and the method comprises treating non-eosinophilic asthma by aerosolizing a pharmaceutical composition comprising encapsulated CBD and administering a unit dose of the aerosolized pharmaceutical composition to a subject in need thereof by inhalation.

When the subject is a mouse, the unit dose can comprise for example, but without limitation, about 0.1, 0.5, 1, 1.5, 2.0, 2.5, 3.0, 3.5, 4.0, 4.5, 5.0, 5.5, 6.0, 6.5, 7.0, 7.5, 8.0, 8.5, 8.0, or about 10 μg of CBD, from about 0.1 to about 10 μg of CBD, from about 0.5 to about 10 μg of CBD, from about 1 to about 10 μg of CBD, from about 1.5 to about 10 μg of CBD, from about 2.0 to about 10 μg of CBD, from about 2.5 to about 10 μg of CBD, from about 3.0 to about 10 μg of CBD, from about 3.5 to about 10 μg of CBD, from about 4.0 to about 10 μg of CBD, from about 4.5 to about 10 μg of CBD, from about 0.1 to about 9.5 μg of CBD, from about 0.1 to about 9.0 μg of CBD, from about 0.1 to about 8.5 μg of CBD, from about 0.1 to about 8.0 μg of CBD, from about 0.1 to about 7.5 μg of CBD, from about 0.1 to about 7.0 μg of CBD, from about 0.1 to about 6.5 μg of CBD, from about 0.1 to about 6.0 μg of CBD, from about 0.1 to about 5.5 μg of CBD, from about 0.5 to about 9.5 μg of CBD, from about 1.0 to about 9.0 μg of CBD, from about 1.5 to about 8.5 μg of CBD, from about 2.0 to about 8.0 μg of CBD, from about 2.5 to about 7.5 μg of CBD, from about 3.0 to about 7.0 μg of CBD, from about 3.5 to about 6.5 μg of CBD, from about 4.0 to about 6.0 μg of CBD, from about 4.5 to about 5.5 μg of CBD. In some aspects, the subject is a mouse and a pharmaceutical composition of the instant disclosure comprises a unit dose form comprising from about 4.5 to about 5.5 μg of CBD.

When the subject is a human, the unit dose can comprise for example, but without limitation, 0.01, 0.05, 0.1, 0.15, 0.20, 0.25, 0.30, 0.35, 0.40, 0.45, 0.50, 0.55, 0.60, 0.65, 0.70, 0.75, 0.80, 0.85, 0.90, 0.95, 1.00, 1.05, 1.10, 1.15, 1.20, 1.25, 1.30, 1.35, 1.40, 1.45, 1.50, 1.55, or about 1.60 mg of CBD, from about 0.01 to about 1.6 mg of CBD, from about 0.05 to about 1.60 mg of CBD, from about 0.10 to about 1.60 mg of CBD, from about 0.15 to about 1.60 mg of CBD, from about 0.20 to about 1.60 mg of CBD, from about 0.25 to about 1.60 mg of CBD, from about 0.30 to about 1.60 mg of CBD, from about 0.35 to about 1.60 mg of CBD, from about 0.40 to about 1.60 mg of CBD, from about 0.45 to about 1.60 mg of CBD, from about 0.50 to about 1.60 mg of CBD, from about 0.55 to about 1.60 mg of CBD, from about 0.60 to about 1.60 mg of CBD, from about 0.65 to about 1.60 mg of CBD, from about 0.70 to about 1.60 mg of CBD, from about 0.01 to about 1.55 mg of CBD, from about 0.01 to about 1.50 mg of CBD, from about 0.01 to about 1.45 mg of CBD, from about 0.01 to about 1.40 mg of CBD, from about 0.01 to about 1.35 mg of CBD, from about 0.01 to about 1.30 mg of CBD, from about 0.01 to about 1.25 mg of CBD, from about 0.01 to about 1.20 mg of CBD, from about 0.01 to about 1.15 mg of CBD, from about 0.01 to about 1.10 mg of CBD, from about 0.01 to about 1.05 mg of CBD, from about 0.01 to about 1.00 mg of CBD, from about 0.01 to about 0.95 mg of CBD, from about 0.01 to about 0.80 mg of CBD, from about 0.05 to about 1.55 mg of CBD, from about 0.10 to about 1.50 mg of CBD, from about 0.15 to about 1.45 mg of CBD, from about 0.20 to about 1.40 mg of CBD, from about 0.25 to about 1.35 mg of CBD, from about 0.30 to about 1.30 mg of CBD, from about 0.35 to about 1.25 mg of CBD, from about 0.40 to about 1.20 mg of CBD, from about 0.45 to about 1.15 mg of CBD, from about 0.50 to about 1.10 mg of CBD, from about 0.55 to about 1.05 mg of CBD, from about 0.60 to about 1.00 mg of CBD, from about 0.65 to about 0.95 mg of CBD, from about 0.70 to about 0.90 mg of CBD, from about 0.70 to about 0.85 mg of CBD, or from about 0.70 to about 0.80 mg of CBD. In some aspects, the subject is a human and a pharmaceutical composition of the instant disclosure comprises a unit dose form comprising from about 0.6 mg to about 0.9 mg of CBD. In some aspects, the subject is a human and a pharmaceutical composition of the instant disclosure comprises a unit dose form comprising from about 0.7 to about 8.0 mg of CBD.

In some aspects, a unit dose of the pharmaceutical spray or aerosol formulation of the instant disclosure is formulated to achieve a desired concentration of cannabinoids in the pulmonary system. In some aspects, the method results in containment of the cannabinoid in lung epithelial cells. In other aspects, the method results in systemic delivery of less than 30% of the cannabinoid into plasma.

In some aspects, a composition of the disclosure is administered as needed, upon development or shortly before development of symptoms. For instance, if the pulmonary inflammatory condition is asthma, the composition can be administered shortly before development of symptoms of asthma or upon development of symptoms of asthma or later.

In some aspects, the composition is administered regularly by following a prescribed treatment schedule. For instance, a composition of the instant disclosure can be administered routinely, at various intervals. For instance, compositions can be administered daily, weekly, monthly, or over a number of months. In some aspects, compositions are administered daily. In other aspects, compositions are administered weekly. In yet other aspects, compositions are administered monthly. Compositions can also be administered every three to six months. As it will be recognized in the art, the duration of treatment can and will vary and can be determined experimentally.

Administration of the compositions described herein can also be carried out as part of a treatment regimen that may include multiple instances of administration of one or more compositions comprising cannabinoids. Such a regimen may be designed as a method of immediately treating a condition and/or as a method of long-term maintenance of the health of a subject after having been treated for a condition (e.g., prevention). For instance, a treatment regimen can be designed to delay the onset of the condition of interest in a subject. It will be appreciated that determination of appropriate treatment regimens is within the skill of practitioners in the art.

It will also be appreciated by those skilled in the art that a composition of the present disclosure may be co-administered with other therapeutic agents before, after, and/or during treatment with a composition of the disclosure. The term “co-administer” refers to administration of more than one active ingredient at the same time, just prior to, or just after the administration of one or more additional therapies. The compounds of the disclosure can be administered alone or can be co-administered to the patient. Co-administration is meant to include simultaneous or sequential administration of the compounds individually or in combination. Dosage amounts and intervals can be adjusted individually to provide levels of the administered compound effective for the particular clinical indication being treated. This will provide a therapeutic regimen that is commensurate with the severity of the individual's disease state.

Accordingly, additional aspects could include any of co-administration, co-formulation, or co-the utilization of the cannabinoid-liposome product with one or more other pulmonary treatment agents. Non-limiting examples of pulmonary treatment agents include steroids or non-steroidal drugs, antifungal or antimicrobial agents, bronchodilators, and any combination thereof. Non-limiting examples of A non-limiting example would include a cannabinoid-liposome product used in conjunction with a bronchodilator. When the cannabinoid-liposome product is used in coordination with a bronchodilator the treatment is more effective than a bronchodilator alone because the bronchodilator only opens the airway up without treating the inflammation. The administration of the cannabinoid-liposome product can more effectively combat the inflammation and can reduce the need for steroids and excessive NSAIDs (all of which have negative side effects), and minimally give patients who have failed front-line therapies another option.

Non-limiting examples of bronchodilators include beta-2 agonists such as salbutamol, salmeterol, formoterol and vilanterol, anticholinergics such as ipratropium, tiotropium, aclidinium, and glycopyrronium, and theophylline. Non-limiting examples of antimicrobial agents include corbomycin, complestatin, isavuconazole, caspofungin, or any combination thereof.

### IV. Kits

A further aspect of the present disclosure provides kits comprising one or more pharmaceutical compositions comprising a cannabinoid detailed above in Section I, wherein each of the compositions if formulated for aerosol administration to a subject. The kit can further comprise a nebulizer suitable for administration of an aerosol comprising the composition of the instant disclosure.

The kits provided herein generally include instructions for carrying out the methods detailed below. Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

The phrase “and/or,” as used herein, should be understood to mean “either or both” of the elements so conjoined, i.e., elements that are conjunctively present in some cases and disjunctively present in other cases.

As used herein, “or” should be understood to have the same meaning as “and/or” as defined above. For example, when separating a listing of items, “and/or” or “or” shall be interpreted as being inclusive, i.e., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including”, “includes”, “having”, “has”, “with”, or variants thereof, are intended to be inclusive similar to the terms “comprising” and “comprises.”

The term “therapeutically effective amount” as used with reference to the present formulation(s) and/or component(s) thereof as described herein refers to the quantity of the formulation(s) and/or component(s) thereof necessary to render the desired therapeutic result. For example, an effective amount is a level effective to treat, cure, or alleviate the symptoms of a disorder for which the therapeutic formulation is being administered. Amounts effective for the particular therapeutic goal sought will depend upon a variety of factors including: the disorder being treated and its severity and/or stage of development/progression; the bioavailability and activity of the specific compound, biologic or pharmaceutical composition used; the route or method of administration and introduction site on the subject; the rate of clearance of the specific compound or biologic and other pharmacokinetic properties; the duration of treatment; inoculation regimen; drugs used in combination or coincident with the specific compound, biologic or composition; the age, body weight, sex, diet, physiology and general health of the subject being treated; and, like factors well known to one of skill in the relevant art. Some variation in dosage will necessarily occur depending upon the condition of the subject being treated, and the physician or other individual administering treatment will, in any event, determine the appropriate dosage for each individual patient.

As used herein, the term “administering” refers to providing a therapeutically effective amount of a formulation and/or components thereof to a patient via any of a number of potential delivery mechanisms including, but not limited to, oral, intravenous, transdermal, topical and/or inhalation, and the like. The formulation and/or components thereof of the present invention can be administered individually, but may also be administered with other compounds, excipients, fillers, binders, carriers or other vehicles selected based upon the chosen route of administration and standard pharmaceutical practice.

As used herein, the term “disorder” refers to a disorder, disease, condition, or other departure from healthy or normal biological activity, and can be used interchangeably. The condition may be caused by any of a number of physical factors. The condition may be caused by sporadic or inheritable genetic abnormalities. The condition may also be caused by non-genetic abnormalities. The condition may also be caused by injuries to a subject from environmental factors. When relating to inflammation, the term disorder is a disorder associated with, caused by, or resulting from inflammation.

As used herein, the terms “treatment” or “treating” refers to arresting, inhibiting, correcting or attempting to arrest or inhibit or correct, the existence, development or progression of a disorder and/or causing, or attempting to cause, the reduction, suppression, regression, or remission of a disorder and/or a symptom thereof. As would be understood by those skilled in the art, various clinical and scientific methodologies and assays may be used to assess the development or progression of a disorder, and similarly, various clinical and scientific methodologies and assays may be used to assess the reduction, regression, or remission of a disorder or its symptoms.

As used herein, the term “treating” refers to: (i) completely or partially inhibiting a disease, disorder or condition, for example, arresting its development; (ii) completely or partially relieving a disease, disorder or condition, for example, causing regression of the disease, disorder and/or condition; or (iii) completely or partially preventing a disease, disorder or condition from occurring in a patient that may be predisposed to the disease, disorder and/or condition, but has not yet been diagnosed as having it. Similarly, “treatment” refers to both therapeutic treatment and prophylactic or preventative measures. In the context of a neurodegenerative disorder, “treat” and “treating” encompass alleviating, ameliorating, delaying the onset of, inhibiting the progression of, or reducing the severity of one or more symptoms associated with the neurodegenerative disorder.

As used herein, the terms “severe asthma (SA),” “neutrophilic asthma,” and “steroid-resistant or unresponsive asthma” can be used interchangeably and can refer to severe asthma, asthma wherein the extent of neutrophilic inflammation is positively associated with the severity of the disease, and steroid-unresponsive asthma.

As various changes could be made in the above-described cells and methods without departing from the scope of the invention, it is intended that all matter contained in the above description and in the examples given below, shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

### Example 1. CBD Liposomes Efficiently Delivers CBD to the Airways

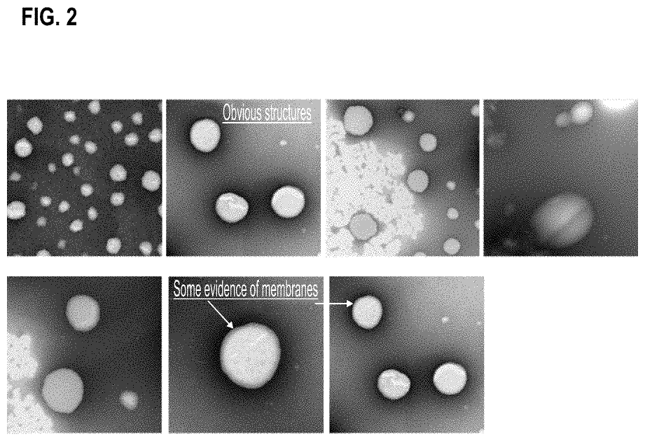

Transmission electron microscopy was used to observe size and polydispersity. Briefly, samples were placed on 150 mesh formvar-coated copper grids treated with poly-1-lysine for approximately 1 hour, then negatively stained with filtered aqueous 2% ammonium molybdate 10.02% BSA, pH 7.0 for 1 minute. Stain was blotted dry from the grids with filter paper, and samples were allowed to dry. Samples were then examined in a JEM 1010 transmission electron microscope (JEOL USA, Peabody, Mass.) at an accelerating voltage of 80 kV. Digital images were obtained using the AMT Imaging System (Advanced Microscopy Techniques Corp, Danvers, Mass.). The diameter of CBD-D liposomes was not uniform and had a Mass Mean Aerodynamic Diameter (MMAD) MAD ranging from about 0.4μ to about 2μ (>70%). FIG. 2 shows electron microscopy images of CBD liposomes showing obvious structures, evidence of membranes and wide distribution of sizes which is important for distribution throughout the lung.

Deposition and transport profiles of aerosolized CBD-DLPC (CBD-D) liposomes were evaluated using a modified Andersen Cascade Impactor (ACI) as an in vitro lung model (FIG. 1). CBD liposomes were aerosolized and administered with an Aerotech II nebulizer, and CBD presence was extracted off ACI plates and quantified by HPLC-MS. CBD concentration in the aerosol was 13±1.7 μg/L. A dose delivered in 30 minutes to a human lung (based on an estimated 0.25 deposition rate) was about 0.75 mg and the dose delivered to a mouse was about 5 μg. The results show that CBD-D could penetrate into and get deposited onto all levels of the lung, including the lower airway.

### Example 2. CBD Liposomes in an Eosinophilic Asthma Model

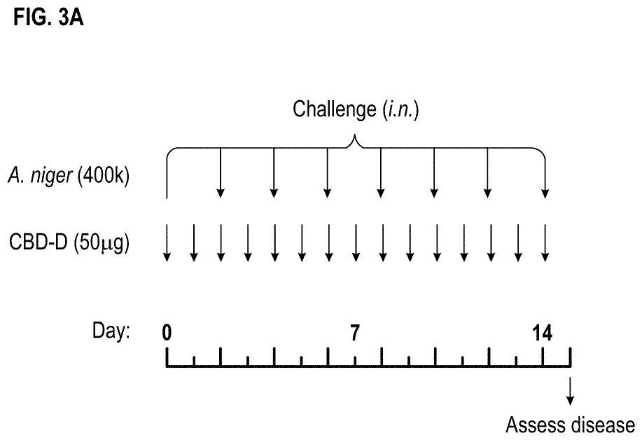

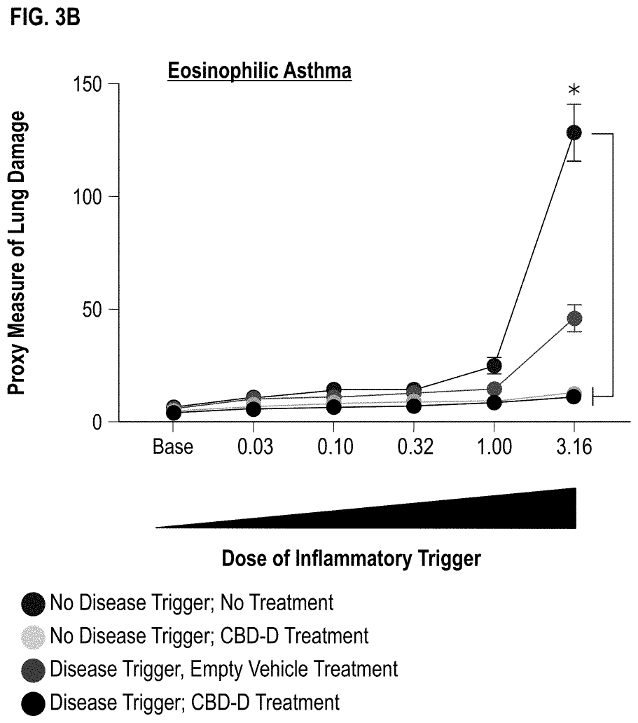

Effect of CBD on inflammation was tested in a mouse eosinophilic asthma model (FIG. 3A). In short, mice were intranasally challenged every other day (q.o.d.) with 400,000 live Aspergillus niger conidia, followed by treatment with CBD liposomes 50 μg aerosolized CBD-D every day (q.d.) Intranasally challenged mice were administered 250 μg aerosolized DLPC liposomes (no CBD) as a control, as well as mice that were not challenged with conidia, administered CBD-D or empty vehicle. Disease was assessed by airway hyperreactivity, inflammatory cell recruitment, and cytokine production. As shown in FIG. 3B, CBD-D does not aid in eosinophilic asthma. In fact administration of CDB in eosinophilic asthma appears to exacerbate disease.

### Example 3. CBD Liposomes in a Neutrophilic Asthma Model

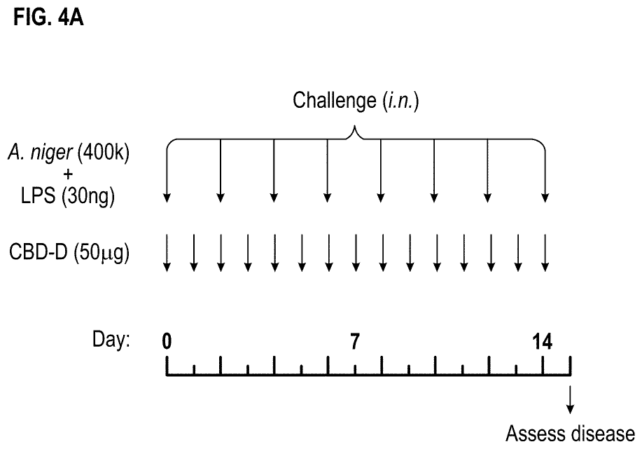

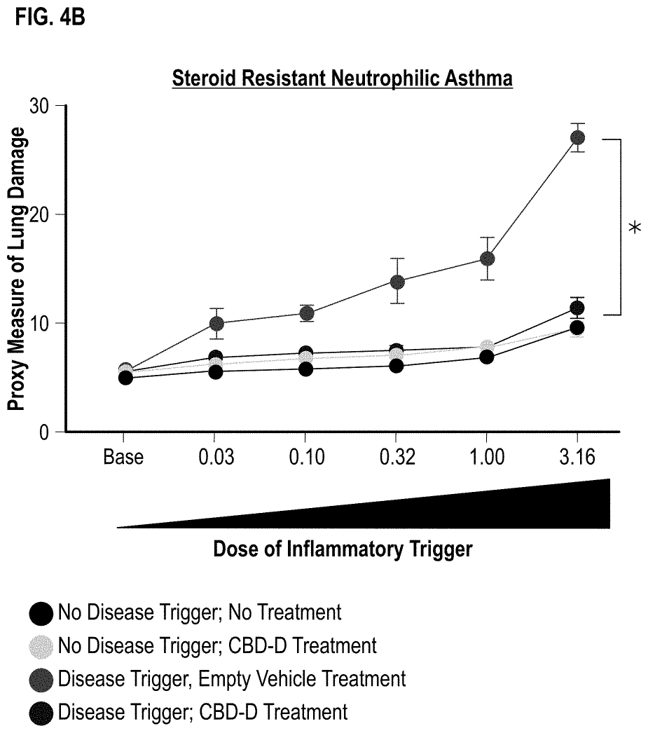

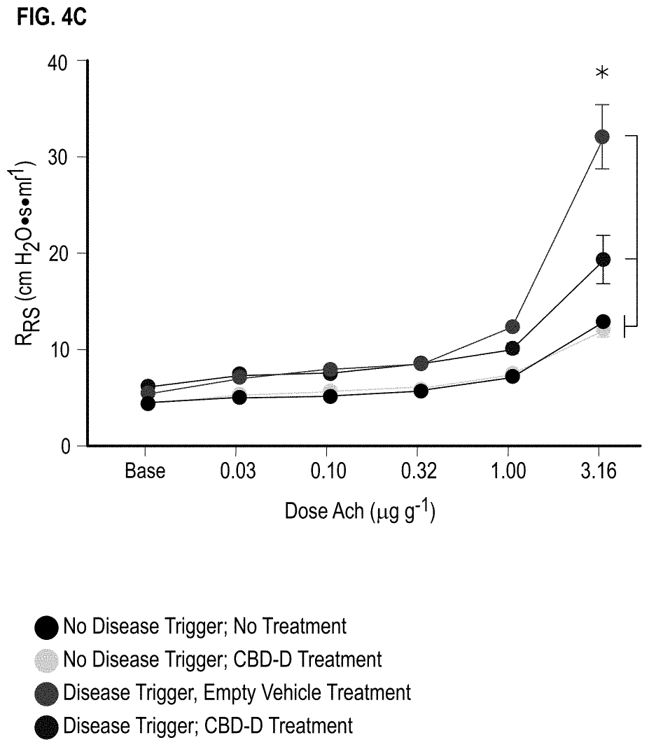

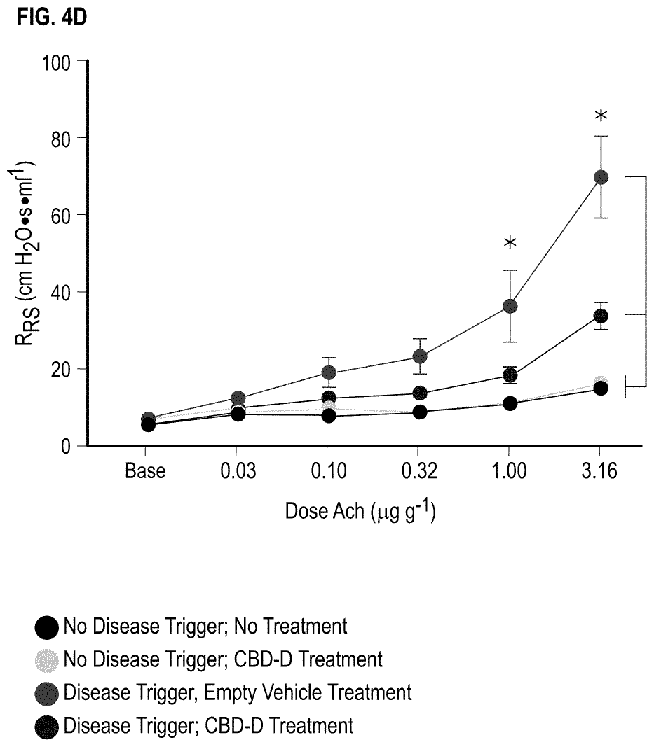

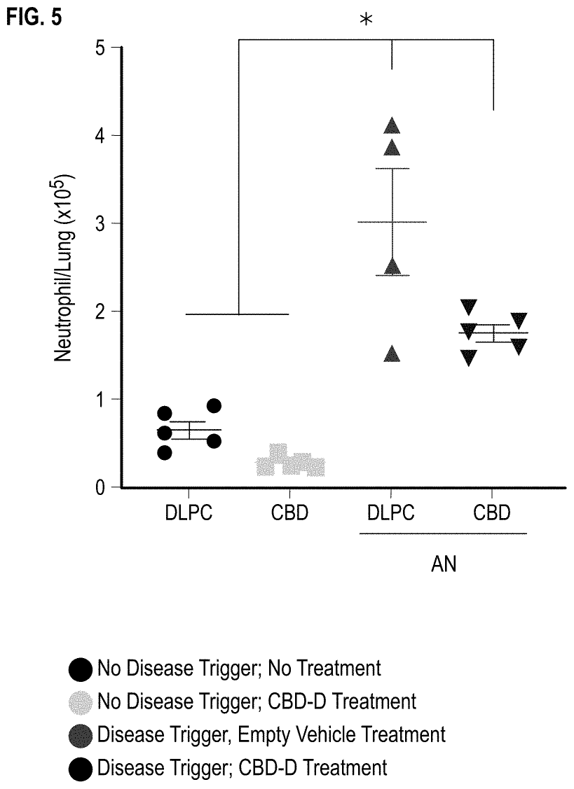

Effect of CBD on inflammation was tested in a mouse neutrophilic asthma model (steroid resistant neutrophilic asthma; FIG. 4A). In short, mice were intranasally challenged every other day (q.o.d.) with 400,000 live Aspergillus niger conidia and 30 ng lipopolysaccharides (LPS), followed by treatment with CBD liposomes 50 μg aerosolized CBD-D every day (q.d.) Intranasally challenged mice were administered 250 μg aerosolized DLPC liposomes (no CBD) as a control, as well as mice that were not challenged with conidia, administered CBD-D or empty vehicle. Disease was assessed by airway hyperreactivity, inflammatory cell recruitment, and cytokine production. As shown in FIG. 4B, CBD-D significantly reduced airway hyperreactivity and damage in neutrophilic asthma. These results were reproducible as shown in FIGS. 4C and 4D. Consistent with these results, CBD-D also significantly reduced neutrophil accumulation in pulmonary spaces of naïve and diseased mice (FIG. 5).

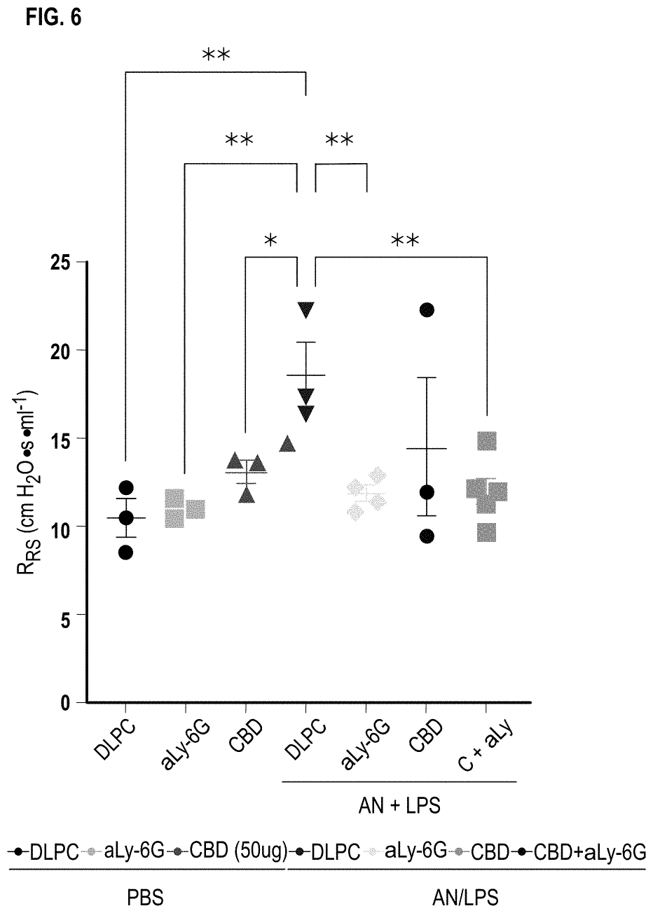

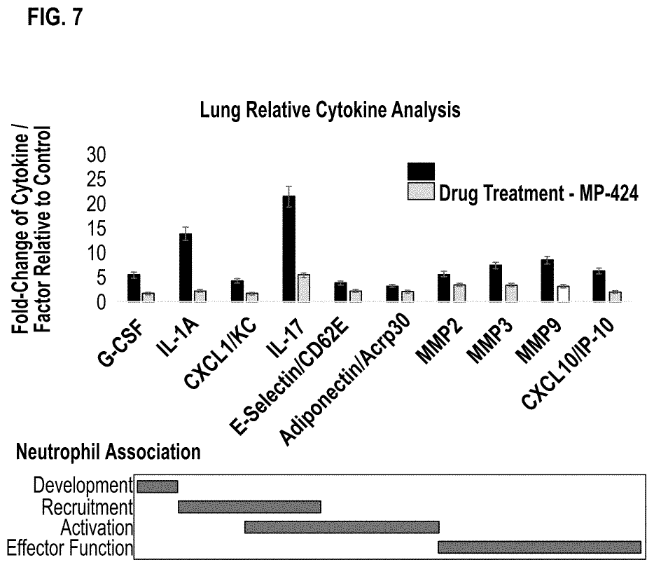

Further confirming the ability of CBD to specifically reduce neutrophilic inflammation using antibody-mediated depletion of neutrophils. In short, neutrophils were depleted using anti-Ly6G monoclonal antibody (MAb) treatment, and the effect of CBD on neutrophil accumulation was tested in the neutrophilic murine model described herein (FIG. 6). The results show that CBD can quell disease in this murine model (black circle), whereas the vehicle alone (DLPC) was unable to prevent airway damage and hyperreactivity in this neutrophil driven asthma model (purple triangles). No inflammation or problems were observed when neutrophils were first depleted (orange diamonds), consistent with this neutrophil-driven asthmatic model. Consistently, when neutrophils are depleted, administration of CBD using CND-D liposomes showed no further improvement since neutrophils that drive the disease were already not present (brown squares). FIG. 7 shows the effect of CBD delivered by CBD-D in challenged mice in the murine neutrophilic model of asthma on cytokine levels (upper panel). The lower panel depicts the function of each cytokine during development of inflammation.

What is claimed is:

1. A method of treating a neutrophilic inflammation or inflammatory disorder in a subject in need thereof, the method comprising administering a therapeutically effective amount of a composition comprising cannabidiol (CBD) to the subject, wherein the composition is encapsulated in a liposomal drug delivery system.

2. The method of claim 1, wherein the inflammation or inflammatory disorder is a neutrophilic pulmonary inflammation or inflammatory disorder.

3. The method of claim 2, wherein the pulmonary inflammation or inflammatory disorder is asthma, chronic obstructive pulmonary disease (COPD), allergic bronchopulmonary aspergillosis, hypersensitivity pneumonia, eosinophilic pneumonia, emphysema, bronchitis, allergic bronchitis bronchiectasis, cystic fibrosis, tuberculosis, hypersensitivity pneumonitis, occupational asthma, sarcoid, reactive airway disease syndrome, interstitial lung disease hypereosinophilic syndrome, rhinitis, sinusitis, exercise-induced asthma, pollution-induced asthma, cough variant asthma, parasitic lung disease, bacterial infections, respiratory syncytial virus (RSV) infection, parainfluenza virus (PIV) infection, rhinovirus (RV) infection or adenovirus infection, or any combination thereof.

4. The method of claim 2, wherein the pulmonary inflammation or inflammatory disorder is neutrophilic asthma.

5. The method of claim 2, wherein the pulmonary inflammation or inflammatory disorder is severe asthma (SA).

6. The method of claim 2, wherein the pulmonary inflammation or inflammatory disorder is steroid-resistant asthma.

7. The method of claim 2, further comprising aerosolizing the composition before administering to pulmonary spaces of the subject.

8. The method of claim 7, wherein the therapeutically effective amount of CBD is in a unit dose form, and the method comprises administering the CBD by aerosolizing the unit dose of the composition to the pulmonary spaces.

9. The method of claim 8, wherein the unit dose is present in and/or delivered from a device suitable for aerosolizing the unit dose into the pulmonary spaces of the subject.

10. The method of claim 9, wherein the unit dose comprises from about 0.6 mg to about 0.9 mg of CBD to a human subject.

11. The method of claim 2, wherein the neutrophilic pulmonary inflammation or inflammatory disorder is SA or steroid-resistant asthma, wherein the method comprises administering a unit dose form of the composition to pulmonary spaces of the subject, and wherein the unit dose comprises from about 0.6 mg to about 0.9 mg of CBD.

12. The method of claim 1, wherein the neutrophilic inflammation or inflammatory disorder is osteoarthritis.

13. The method of claim 12, wherein the method comprises orally administering from about 90 mg to about 110 mg of CBD to a human subject.

14. The method of claim 13, wherein the method comprises administering about 20 mg/kg of CBD to a human subject per day.

15. A method of treating a neutrophilic pulmonary inflammation or inflammatory disorder in a subject in need thereof, the method comprising administering a composition comprising a therapeutically effective amount of cannabidiol (CBD) encapsulated in a liposomal drug delivery system, wherein the therapeutically effective amount comprises from about 0.6 mg to about 0.9 mg of CBD.

16. A method of treating a neutrophilic asthma in a subject in need thereof, the method comprising administering a composition comprising a therapeutically effective amount of cannabidiol (CBD) encapsulated in a liposomal drug delivery system, wherein the therapeutically effective amount comprises from about 0.6 mg to about 0.9 mg of CBD.
